SEQUENCE LISTING (8 sequences)

SEQ ID NO: 1 - DNA, 32 bp, Artificial sequence
  DNA Primer
gggagctcgagataagaattactagttaattc

SEQ ID NO: 2 - DNA, 35 bp, Artificial sequence
  DNA Primer
aagcggccgcccatgcatttcacctattagttatc

SEQ ID NO: 3 - DNA, 34 bp, Artificial sequence
  DNA Primer
aagcggccgcatcgaaggtcgtcatcaccatcac

SEQ ID NO: 4 - DNA, 28 bp, Artificial sequence
  DNA Primer
gtttatatataagatgttaataacacag

SEQ ID NO: 5 - DNA, 34 bp, Artificial sequence
  DNA Primer
aaatgcatggcaaaatttgaggacaaagttgacc

SEQ ID NO: 6 - DNA, 39 bp, Artificial sequence
  DNA Primer
aaagatctttagtgatggtgatggtgatgacgaccttcg

SEQ ID NO: 7 - DNA, 34 bp, Artificial sequence
  DNA Primer
aaatgcatggctgacaagattgatttatatgatg

SEQ ID NO: 8 - DNA, 38 bp, Artificial sequence
  DNA Primer
aaagatcttagtgatggtgatggtgatgacgaccttcg

(19) United States
(12) Patent Application Publication
(10) Pub. No.: US 2016/0168598 A1
(43) Pub. Date: Jun. 16, 2016

(54) GENETICALLY MODIFIED ORGANISMS

(71) Applicant: UNIVERSITY OF WASHINGTON, Seattle, WA (US)

(72) Inventors: JOHN ARTHUR LEIGH, Seattle, WA (US); THOMAS JOSEPH LIE, Seattle, WA (US)

(21) Appl. No.: 14/963,836

(22) Filed: Dec. 9, 2015

Related U.S. Application Data
(60) Provisional application No. 62/090,159, filed on Dec. 10, 2014.

(51) Int. Cl. C12P 7/16; C10L 1/02; C12N 9/10; C12P 7/04; C12P 7/06

(52) U.S. Cl. CPC C12P 7/16; C12P 7/04; C12P 7/06; C12N 9/13; C12N 9/1007; C10L 1/02; C12Y 208/04001; C12Y 201/0109; C10L 2200/0469

(57) ABSTRACT

Provided herein is technology relating to genetically modified organisms and particularly, but not exclusively, to compositions comprising one or more genetically modified microorganisms, and related methods and systems, for producing liquid fuels from alkanes such as methane.

This application claims priority to U.S. provisional patent application Ser. No. 62/090,159, filed Dec. 10, 2014, which is incorporated herein by reference in its entirety.

### STATEMENT REGARDING FEDERALLY SPONSORED RESEARCH OR DEVELOPMENT

This invention was made with government support under DE-AR0000426 awarded by the Department of Energy. The government has certain rights in the invention.

### FIELD

Provided herein is technology relating to genetically modified organisms and particularly, but not exclusively, to compositions comprising one or more genetically modified microorganisms, and related methods and systems, for producing liquid fuels from alkanes such as methane.

### BACKGROUND

Natural gas is abundant throughout the United States and is often used for heating, cooking, and electrical power generation. Natural gas comprises primarily methane, an energy-rich compound not widely used for transportation. Currently, the use of methane for fuel production is limited by the paucity of commercially feasible approaches to convert methane into liquid fuel. The current state-of-the-art process for the conversion of natural gas to liquid fuels utilizes a chemical synthetic process (Fischer-Tropsch chemistry), which is expensive and inefficient. Further, current processes have numerous processing requirements, environmental conditions (e.g., high pressure and temperature), and safety concerns. Accordingly, alternative approaches are needed.

### SUMMARY

Thus, to take advantage of natural gas resources, new biological processes provided by special microorganisms called “biocatalysts” are needed to transform methane into liquid fuels. In addition to commercial advantages, embodiments of these biocatalyst technologies provide environmental advantages because they are carbon neutral or have less environmental impact relative to traditional fuels.

The present technology provides efficient and cost-effective methods for biosynthetic production of methanol and other alcohol products from alkanes using genetically engineered microorganisms. In particular, provided herein is technology related to genetically engineered microorganisms that provide for the anaerobic oxidation of methane, which is a fundamental step in creating a liquid fuel from natural gas in some synthetic schemes. In particular embodiments, the technology provides for the bioconversion of methane to methanol in a pathway coupled to sulfate reduction. The technology integrates these metabolic pathways into a single, novel, genetically engineered microorganism comprising recently discovered enzymes from independent and distinct organisms that naturally catalyze these processes.

In contrast to current approaches for methane oxidation that require the addition of oxygen and energy in the form of heat, which is inefficient and costly, embodiments of the technology provided herein relate to a genetically engineered microorganism that oxidizes methane anaerobically without the need for these additional inputs of energy. In some embodiments, the genetically engineered organisms provide a way to produce butanol, which is compatible with gasoline at any ratio and can also be used as a pure fuel in existing combustion engines without modifications.

Accordingly, embodiments of this technology provide for greater utilization of natural gas reserves as a fuel source, thus significantly reducing demand for foreign oil and reducing harmful emissions associated with conventional fuel technologies. Further, Expanding natural gas resources via bioconversion to liquid fuels will contribute tens of billions of dollars to the economy while reducing or stabilizing transport fuel prices.

Provided herein are embodiments of technologies related to a genetically engineered (e.g., non-natural) microorganism comprising a host microorganism and a heterologous methyl coenzyme M reductase. The technology is not limited in the host organism that is provided to genetically engineer the non-natural organism. The host organism is typically, but not necessarily, a genetically tractable (e.g., culturable under laboratory conditions and manipulable by molecular biological techniques) organism. The host organism may be a member of the domain Bacteria, the domain Eukarya, or the domain Archaea. In some embodiments of the technology, the host microorganism is from the domain Archaea. In some embodiments, the host organism is a methanogen and in some embodiments the host organism is a methanotroph. In particular embodiments, the host microorganism is from the genus Methanococcus and in some particular embodiments the host microorganism is from the genus Methanosarcina. In exemplary embodiments, the host microorganism is Methanococcus maripaludis.

Further, the technology is not limited in the source organism from which heterologous genes, operons, proteins, pathways, activities, etc. are obtained for use in the genetically engineered microorganism. For instance, in some embodiments, the heterologous methyl coenzyme M reductase is from a source microorganism that is an anaerobic methanotroph; in some embodiments, the heterologous methyl coenzyme M reductase is from a source microorganism that is an aerobic methanotroph. The source organism may be a member of the domain Bacteria, the domain Eukarya, or the domain Archaea. Accordingly, in some embodiments, the heterologous methyl coenzyme M reductase is from a source microorganism that is from the domain Archaea.

While aerobic methane oxidation is well-known, recent discoveries have demonstrated that methane is also oxidized anaerobically, e.g., in marine sediments by microbial communities comprising methanotrophic archaea (e.g., ANME-1, ANME-2, or ANME-3) and sulfate-reducing and/or nitrate-reducing bacteria. In particular, the enzyme methyl coenzyme M reductase (MCR) has activity not only for the anaerobic synthesis of methane but also for the oxidation of methane (AOM). Some embodiments are related to the use of a MCR from a methanogen and/or from an anaerobic methane oxidizer. Thus, in particular embodiments, the heterologous methyl coenzyme M reductase is from a source microorganism that is an anaerobic methanotroph, e.g., a group 1 anaerobic methanotroph (ANME-1), a group 2 anaerobic methanotroph (ANME-2), or a group 3 anaerobic methanotroph (ANME-3). In some embodiments, the heterologous methyl coenzyme M reductase is from a source microorganism that is from the genus Methanothermobacter. Particular embodiments provide that the heterologous methyl coenzyme M reductase is from a source microorganism that is Methanothermobacter marburgensis.

In some embodiments of the technology the genetically engineered microorganism further comprises a methanol:coenzyme M methyltransferase, e.g., a heterologous methanol:coenzyme M methyltransferase. For example, in some embodiments the genetically engineered microorganism comprises a methanol:coenzyme M methyltransferase that is from a source organism from the genus Methanosarcina or Methanosphaera, e.g., a source organism that is Methanosarcina barkeri or Methanosphaera stadtmanae.

The technology is not limited in the genes, operons, proteins, protein complexes, etc. (either known or unknown) that provide the methyl coenzyme M reductase and/or methyl coenzyme M reductase activity. For instance, in some embodiments the methyl coenzyme M reductase or the heterologous methyl coenzyme M reductase is encoded by mcrB, mcrD, mcrC, mcrG, and/or mcrA genes. Accordingly, in some embodiments, the genetically engineered microorganism comprises a vector comprising mcrB, mcrD, mcrC, mcrG, and/or mcrA genes. In some embodiments, the mcrB, mcrD, mcrC, mcrG, and/or mcrA genes are organized in an operon; accordingly, in some embodiments the genetically engineered microorganism comprises an mcr operon. In some embodiments, the genetically engineered microorganism comprises a vector comprising an mcr operon. In particular embodiments, the heterologous methyl coenzyme M reductase is encoded by Methanothermobacter marburgensis mcrB, mcrD, mcrC, mcrG, and/or mcrA genes. And, in particular embodiments, the heterologous methyl coenzyme M reductase is encoded by mcrB, mcrD, mcrC, mcrG, and/or mcrA genes from an anaerobic methanotroph, e.g., by mcrB, mcrD, mcrC, mcrG, and/or mcrA genes from a group 1 anaerobic methanotroph (ANME-1), a group 2 anaerobic methanotroph (ANME-2), or a group 3 anaerobic methanotroph (ANME-3).

In other embodiments, the methanol:coenzyme M methyltransferase is encoded by mtaA, mtaB, and mtaC genes. Accordingly, in some embodiments the genetically engineered microorganism comprises a vector comprising an mta operon and in some embodiments the genetically engineered microorganism comprises a vector comprising mtaA, mtaB, and/or mtaC genes.

In some embodiments, a vector integrates into the genome of the genetically engineered microorganism (e.g., the genetically engineered microorganism comprises an “integrated vector” or a “genome-integrated vector”) and in some embodiments the vector remains separate (e.g., as an episomal nucleic acid) from the host microorganism genome (e.g., the genetically engineered microorganism comprises an “episome” or an “episomal genetic component” or an “episomal vector”). Accordingly, in some embodiments, the genetically engineered microorganism comprises heterologous nucleic acids integrated into its genome and in some embodiments the genetically engineered microorganism comprises heterologous nucleic acids in an episomal genetic component.

As discussed herein, the technology relates in some embodiments to expressing an MCR (e.g., from Methanothermobacter marburgensis, ANME-1, ANME-2, and/or ANME-3) in a genetically tractable methanogen host microorganism to provide a genetically engineered microorganism comprising one or more metabolic pathways for converting an alkane (e.g., methane) to a fuel (e.g., an alcohol such as methanol and/or butanol). In some embodiments, the pathway for the conversion of an alkane to a fuel is thermodynamically coupled to another pathway to provide a system of pathways that is thermodynamically favorable. For instance, in some embodiments the pathway for the conversion of an alkane to a fuel is thermodynamically coupled to a dissimilatory sulfate reduction pathway to provide a “natural gas to liquid fuel” process that is thermodynamically favorable.

Accordingly, some embodiments of the technology provide a genetically engineered microorganism as described above further comprising a heterodisulfide reductase, e.g., in some embodiments a heterologous heterodisulfide reductase. In some embodiments, the genetically engineered microorganism further comprises an ATP sulfurylase, an APS reductase, a sulfite reductase, and/or a hydrogenase, e.g., a heterologous ATP sulfurylase, a heterologous APS reductase, a heterologous sulfite reductase, and/or a heterologous hydrogenase.

In some embodiments, other enzymes and/or activities are provided in the genetically engineered microorganism to provide useful functions and/or activities. For example, in some embodiments the genetically engineered microorganism further comprises increased expression of glyceraldehyde-3-phosphate:ferredoxin oxidoreductase and/or a heterologous glyceraldehyde-3-phosphate:ferredoxin oxidoreductase; in some embodiments, the genetically engineered microorganism further comprises a sulfite reductase gene and/or a heterologous sulfite reductase gene.

In sum, the technology provides, in some embodiments, a genetically engineered microorganism comprising a metabolic pathway that converts methane to methanol. In some particular embodiments, the genetically engineered microorganism comprises a first metabolic pathway that converts methane to methyl-coenzyme M and a second metabolic pathway that converts methyl-coenzyme M to methanol. And, in some embodiments the genetically engineered microorganism comprises a first metabolic pathway that converts methane to methanol coupled to a second metabolic pathway to provide a thermodynamically favorable coupled system of the first and second metabolic pathways. For example, in some embodiments, the genetically engineered microorganism comprises a first metabolic pathway that converts methane to methanol coupled to a second metabolic pathway that reduces sulfate, e.g., a second metabolic pathway that converts sulfate to sulfide.

Various embodiments are related to a genetically engineered microorganism comprising a component (e.g., a protein and/or protein complex) that provides for electron transport between other components of the metabolic pathways. For example, in some embodiments the genetically engineered microorganism further comprises a QmoABC complex, e.g., in some embodiments a heterologous QmoABC complex.

In some embodiments, the genetically engineered microorganism further comprises a pathway for sulfite reduction comprising an assimilatory sulfite reductase, e.g., in some embodiments the genetically engineered microorganism further comprises a pathway for sulfite reduction comprising an assimilatory sulfite reductase Fsr or, in some embodiments, a pathway for sulfite reduction comprising a heterologous assimilatory sulfite reductase Fsr. In some embodiments, the assimilatory sulfite reductase Fsr is from a source organism that is Methanothermococcus thermolithotrophicus, Methanococcus aeolicus, or Methanocaldococcus jannaschii.

Some embodiments provide a pathway for the production of a fuel such as an alcohol (e.g., methanol). Some embodiments provide for the conversion of one fuel (e.g., an alcohol such as methanol) into another fuel (e.g., an alcohol such as butanol, propanol, etc.). Accordingly, some embodiments provide a genetically engineered microorganism that comprises a pathway for converting a first alcohol into a second alcohol. Some embodiments provide a genetically engineered microorganism comprising a pathway for converting methanol into butanol; thus, some embodiments provide a genetically engineered microorganism comprising a Wood-Ljungdahl pathway, e.g., a heterologous Wood-Ljungdahl pathway. For example, some embodiments provide a genetically engineered microorganism comprising a Wood-Ljungdahl pathway from a source organism that is from the genus Clostridium.

Thus, in some embodiments the technology provides a genetically engineered microorganism comprising a pathway for production of butanol (e.g., from an alkane (e.g., methane); e.g., from an alcohol (e.g., methanol)). In some exemplary embodiments, the genetically engineered microorganism comprises a pathway for production of butanol from acetyl-CoA. And, in further embodiments the genetically engineered microorganism comprises an inactivated gene encoding methyl-tetrahydromethanopterin reductase (e.g., a knockout of the gene encoding methyl-tetrahydromethanopterin reductase). In some embodiments, the genetically engineered microorganism comprises an inactivated mer gene (e.g., the genetically engineered microorganism is mer⁻).

Additional embodiments provide a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein. In some embodiments, the system converts a composition comprising an alkane (e.g., methane) or a composition comprising a mixture of alkanes to a fuel. In some embodiments, the fuel is an alcohol or a mixture of alcohols (e.g., methanol or butanol or a mixture comprising methanol and butanol). In some embodiments, the composition comprises natural gas.

Further system embodiments provide a system for converting a composition comprising an alkane to a fuel, the system comprising a biocatalyst having activity for anaerobic oxidization of the alkane. For example, in some embodiments the biocatalyst comprises a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein.

Some embodiments are related to compositions, e.g., a composition comprising a fuel. In some embodiments, the technology provides a fuel produced by a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein. Some embodiments provide a fuel mixture, e.g., a fuel comprising gasoline and a fuel produced by a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein.

Related embodiments provide methods for producing a fuel from a composition comprising an alkane (e.g., a composition comprising methane such as natural gas or a composition derived from natural gas), the method comprising providing a composition comprising an alkane and exposing the composition comprising an alkane to a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase). Some method embodiments further comprise isolating the fuel. In some embodiments, the fuel comprises methanol and/or butanol. Some embodiments further comprise mixing the fuel (e.g., comprising methanol and/or butanol) with gasoline.

In other embodiments, the technology provides a method for producing a fuel from a composition comprising an alkane, the method comprising providing a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein; and exposing the genetically engineered microorganism, the cell-free fraction derived from the genetically engineered microorganism, or the system to a composition comprising an alkane.

Embodiments provide a fuel produced by a method comprising the steps of providing a composition comprising an alkane; and exposing the composition comprising an alkane to a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein.

Embodiments provide a fuel mixture comprising gasoline and a fuel produced by a method comprising the steps of providing a composition comprising an alkane; and exposing the composition comprising an alkane to a genetically engineered microorganism as described herein (e.g., a genetically engineered (e.g., non-natural) microorganism comprising a host microorganism and a heterologous methyl coenzyme M reductase), a cell-free fraction derived from a genetically engineered microorganism as described herein, or a system for converting a composition comprising an alkane to a fuel, the system comprising a genetically engineered microorganism as described herein (e.g., a genetically engineered (e.g., non-natural) microorganism comprising a host microorganism and a heterologous methyl coenzyme M reductase) or a cell-free fraction derived from a genetically engineered microorganism as described herein.

The systems provided herein comprise, in some embodiments, a gas phase bioreactor and/or a fluidized bed bioreactor. In some embodiments, the systems comprise a source of the composition comprising an alkane, e.g., the systems comprise a source of natural gas. Some embodiments of systems comprise a conduit in fluid communication with a bioreactor comprising the engineered microorganism or a cell-free fraction derived from a genetically engineered microorganism. Further, some embodiments comprise a source of the composition comprising an alkane in fluid communication with a bioreactor comprising the engineered microorganism or the cell-free fraction derived from a genetically engineered microorganism. Some embodiments comprise a collection unit for collecting a composition comprising the fuel, e.g., some embodiments comprise a condenser or distillation unit for collecting a composition comprising the fuel. In some embodiments, the genetically engineered microorganism or the cell-free fraction derived from a genetically engineered microorganism is immobilized on a solid matrix.

In some embodiments the technology relates to cultures comprising a genetically engineered microorganism as described herein. For example, some embodiments provide a culture of 10 ml or more (e.g., 10 ml, 25 ml, 50 ml, 100 ml, 200 ml, 250 ml, 500 ml, 750 ml, 1 L, 2 L, 2.5 L, 5 L, or 10 L). Embodiments provide a culture (e.g., having a volume of more than 10 ml) comprising greater than 10⁴ genetically engineered microorganisms per milliliter (e.g., more than 10⁴/ml, more than 10⁵/ml, more than 10⁶/ml, more than 10⁷/ml).

Some embodiments provide for production scale cultures larger than 10 L. In some embodiments, a culture of 10 ml or more is used as a starter culture to produce a production scale culture.

Embodiments provide a culture of 10 ml or more comprising a genetically engineered microorganism for the anaerobic oxidation of methane. Embodiments provide a culture having a volume of 10 ml or more comprising a genetically engineered (e.g., non-natural) microorganism comprising a host microorganism and a heterologous methyl coenzyme M reductase. Some embodiments provide a culture having a volume of 10 ml or more comprising a host organism that is a methanogen or a methanotroph. In particular embodiments, a culture having a volume of 10 ml or more comprises a host microorganism from the genus Methanococcus or from the genus Methanosarcina. In some embodiments, a culture having a volume of 10 ml or more comprises a genetically engineered microorganism comprising a host that is Methanococcus maripaludis. Some embodiments provide a culture having a volume of 10 ml or more comprising a genetically engineered microorganism comprising a heterologous methyl coenzyme M reductase that is from a source microorganism that is an anaerobic methanotroph or that is an aerobic methanotroph. Some embodiments provide a culture having a volume of 10 ml or more comprising a genetically engineered microorganism that comprises a host that is Methanococcus maripaludis and a heterologous methyl coenzyme M reductase from a source microorganism that is Methanothermobacter marburgensis or an anaerobic methanotroph such as a group 1 anaerobic methanotroph (ANME-1), a group 2 anaerobic methanotroph (ANME-2), or a group 3 anaerobic methanotroph (ANME-3).

Some embodiments provide a culture of 10 ml or more comprising a genetically engineered microorganism for biosynthetic production of methanol and other alcohol products from alkanes. Some embodiments provide a culture of 10 ml or more comprising a genetically engineered microorganism for the anaerobic oxidation of methane. Some embodiments provide a culture of 10 ml or more comprising a genetically engineered microorganism expressing an MCR (e.g., from ANME-1 and/or ANME-2) in a genetically tractable methanogen host microorganism (e.g., Methanococcus maripaludis) to provide a genetically engineered microorganism comprising one or more metabolic pathways for converting an alkane (e.g., methane) to a fuel (e.g., an alcohol such as methanol and/or butanol).

Embodiments provide a culture (e.g., having a volume of more than 10 ml) comprising greater than 10⁴ genetically engineered microorganisms per milliliter for the anaerobic oxidation of methane. Embodiments provide a culture (e.g., having a volume of more than 10 ml) comprising greater than 10⁴ genetically engineered microorganisms per milliliter comprising a host microorganism and a heterologous methyl coenzyme M reductase. Some embodiments provide a culture (e.g., having a volume of more than 10 ml) comprising greater than 10⁴ genetically engineered microorganisms per milliliter that are methanogens or methanotrophs. In particular embodiments, a culture (e.g., having a volume of more than 10 ml) comprising greater than 10⁴ genetically engineered microorganisms per milliliter comprises a host microorganisms from the genus Methanococcus or from the genus Methanosarcina. In some embodiments, a culture (e.g., having a volume of more than 10 ml) comprising greater than 10⁴ genetically engineered microorganisms per milliliter comprises genetically engineered microorganisms comprising a host that is Methanococcus maripaludis. Some embodiments provide a culture (e.g., having a volume of more than 10 ml) comprising greater than 10⁴ genetically engineered microorganisms per milliliter comprising genetically engineered microorganisms comprising a heterologous methyl coenzyme M reductase that is from a source microorganism that is an anaerobic methanotroph or that is an aerobic methanotroph. Some embodiments provide a culture (e.g., having a volume of more than 10 ml) comprising greater than 10⁴ genetically engineered microorganisms per milliliter that comprises a host that is Methanococcus maripaludis and a heterologous methyl coenzyme M reductase from a source microorganism that is Methanothermobacter marburgensis or an anaerobic methanotroph such as a group 1 anaerobic methanotroph (ANME-1), a group 2 anaerobic methanotroph (ANME-2), or a group 3 anaerobic methanotroph (ANME-3).

Embodiments provide a culture (e.g., having a volume of more than 10 ml) comprising greater than 10⁴ genetically engineered microorganisms per milliliter for biosynthetic production of methanol and other alcohol products from alkanes. Some embodiments provide a culture (e.g., having a volume of more than 10 ml) comprising greater than 10⁴ genetically engineered microorganisms per milliliter for the anaerobic oxidation of methane. Some embodiments provide a culture (e.g., having a volume of more than 10 ml) comprising greater than 10⁴ genetically engineered microorganisms per milliliter expressing an MCR (e.g., from a source microorganism that is Methanothermobacter marburgensis or an anaerobic methanotroph such as a group 1 anaerobic methanotroph (ANME-1), a group 2 anaerobic methanotroph (ANME-2), or a group 3 anaerobic methanotroph (ANME-3)) in a genetically tractable methanogen host microorganism (e.g., Methanococcus maripaludis). Embodiments provide a culture (e.g., having a volume of more than 10 ml) comprising greater than 10⁴ genetically engineered microorganisms per milliliter to provide a culture for converting an alkane (e.g., methane) to a fuel (e.g., an alcohol such as methanol and/or butanol).

In some embodiments, the technology finds use in research. For example, in some embodiments the technology provides a culturable microorganism comprising an exogenous nucleic acid encoding an enzyme with coenzyme M reductase activity for study and analysis in a laboratory environment, e.g., to provide a system to analyze the anaerobic oxidation of methane. In some embodiments, the technology provides a culturable microorganism comprising an exogenous nucleic acid encoding an enzyme with coenzyme M reductase activity for computational modeling of metabolic flux in an organism. In some embodiments, a genetically engineered microorganism comprising a host that is Methanococcus maripaludis and a heterologous methyl coenzyme M reductase from a source microorganism that is Methanothermobacter marburgensis or an anaerobic methanotroph such as a group 1 anaerobic methanotroph (ANME-1), a group 2 anaerobic methanotroph (ANME-2), or a group 3 anaerobic methanotroph (ANME-3) finds use in research as a platform into which to introduce and study additional metabolic capabilities.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a diagram of an engineered metabolic scheme for the conversion of an alkane (e.g., methane: CH₄) to an alcohol (e.g., methanol: CH₃—OH). In the metabolic system shown in FIG. 1, conversion of methane to methanol (Module 1) is coupled to the conversion of sulfate to sulfide (Module 2). MCR: methyl-coenzyme M reductase; CoBSSCoM: heterodisulfide of coenzyme M and coenzyme B; CoBSH, coenzyme B; CoMSH: coenzyme M; CH₃-SCoM: methyl-coenzyme M; MtaABC: methanol:coenzyme M methyltransferase enzyme; HDR, heterodisulfide reductase enzyme.

FIG. 2 is a diagram showing an engineered metabolic scheme for coupling the oxidation of methane to the reduction of sulfate (SO₄²⁻). Redox potentials are shown under standard conditions (1 atm H₂). Fd: ferredoxin; Fd_{red}: ferredoxin in reduced state; Fdₒₓ: ferredoxin in oxidized state; VHU: hydrogenase; EHA/B: hydrogenase; SAT: ATP sulfurylase; APR: APS reductase; DSR: sulfite reductase.

FIG. 3 is a diagram showing an engineered metabolic scheme for converting methanol to butanol. adh: bifunctional acetaldehyde/alcohol dehydrogenase; thl: acetyl-CoA acetyltransferase; hbd: 3-hydroxybutyryl-CoA dehydrogenase; crt: 3-hydroxybutyryl-CoA dehydratase; bcd: butyryl-CoA dehydrogenase; H₄MPT: tetrahydromethanopterin; CH₃-H₄MPT: methyl-tetrahydromethanopterin; MtaABC: methanol:coenzyme M methyltransferase enzyme; mtrEDCBAFG: methyltransferase.

FIG. 4 is a drawing showing a map of an exemplary plasmid vector based on the pWLG vector and comprising the mcrB, mcrD, mcrC, mcrG, and mcrA genes from Methanothermobacter marburgensis. The plasmid comprises a neomycin resistance marker (NeoR) and an ampicillin resistance marker (AmpR) for use in molecular biological manipulation of the plasmid vector (e.g., transformation, production, and selection/counterselection of transformed organisms).

FIG. 5 is a schematic drawing showing a Methanosarcina acetivorans methanol methyltransferase synthetic operon. The operon comprises four Mta genes (mtaC1 (MA0456); mtaB1 (MA0455)+histidine tag; mtaA1 (MA4379); and MA4380 redox activator protein) with intergenic sequences from Methanococcus voltae (Mvo1129 intergenic; Mvo1130 intergenic; and Mvo1131 intergenic). The genes are codon-optimized and the mtaB1 subunit contains a histidine tag on the C-terminal end.

FIG. 6A is a western blot showing high expression of the histidine-tagged Methanosarcina acetivorans MtaB protein in Methanococcus maripaludis. HdrA is a positive control having a previously determined high level of expression. FIG. 6B is an image of a polyacrylamide gel showing the purification of MtaB and MtaC from M maripaludis. A M. maripaludis cell extract was produced and incubated with Ni-NTA agarose; bound proteins were eluted with imidazole. CbiX is a native protein that naturally contains a run of histidine residues and serves as an internal standard.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to genetically modified organisms and particularly, but not exclusively, to compositions comprising one or more genetically modified microorganisms, and related methods and systems, for producing liquid fuels from alkanes such as methane.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. Thus, for example, reference to “a polynucleotide” includes a plurality of such polynucleotides and reference to “the microorganism” includes reference to one or more microorganisms, and so forth. The meaning of “in” includes “in” and “on.”

The term “microorganism” includes prokaryotic and eukaryotic microbial species from the domains Archaea, Bacteria, and Eukarya, the latter including yeast and filamentous fungi, protozoa, algae, or higher Protista. The terms “microbial cells” and “microbes” are used interchangeably with the term “microorganism”.

The term “prokaryotes” refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The terms “bacteria” and “bacterium” and “archaea” and “archaeon” refer to prokaryotic organisms of the domain Bacteria and Archaea in the three-domain system (see Woese C R, et al., Proc Natl Acad Sci USA 1990, 87: 4576-79).

The term “Archaea” refers to a taxonomic domain of organisms typically found in unusual environments and distinguished from the rest of the prokaryotes by several criteria, including the number of ribosomal proteins and the lack of muramic acid in cell walls. On the basis of small subunit rRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (e.g., no murein in cell wall, ester-linked membrane lipids, etc.), these prokaryotes exhibit unique structural or biochemical attributes which adapt them to their particular habitats. The Crenarchaeota consist mainly of hyperthermophilic sulfur-dependent prokaryotes and the Euryarchaeota contain the methanogens and extreme halophiles.

The term “Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity et al. (2007) The Taxonomic Outline of Bacteria and Archaea. TOBA Release 7.7, March 2007. Michigan State University Board of Trustees).

The term “species” is defined as a collection of closely related organisms with greater than 97% 16S ribosomal RNA sequence homology and greater than 70% genomic hybridization and sufficiently different from all other organisms so as to be recognized as a distinct unit.

The term “strain” as used herein in reference to a microorganism describes an isolate of a microorganism considered to be of the same species but with a unique genome and, if nucleotide changes are non-synonymous, a unique proteome differing from other strains of the same organism. Strains may differ in their non-chromosomal genetic complement. Typically, strains are the result of isolation from a different host or at a different location and time, but multiple strains of the same organism may be isolated from the same host.

As used herein, the term “naturally occurring” as applied to a nucleic acid, an enzyme, a cell, or an organism, refers to a nucleic acid, enzyme, cell, or organism that is found in nature. For example, a polypeptide or polynucleotide sequence that is present in an organism that can be isolated from a source in nature and that has not been intentionally modified by a human in the laboratory is naturally occurring.

As used herein, the term “non-naturally occurring” as applied to a nucleic acid, an enzyme, a cell, or an organism refers to a nucleic acid, an enzyme, a cell, or an organism that has at least one genetic alteration not normally found in the naturally occurring nucleic acid, enzyme, cell, or organism. Genetic alterations include, for example, modifications introducing expressible nucleic acids encoding metabolic polypeptides, other nucleic acid additions, nucleic acid deletions, and/or other functional disruption of the microbial genetic material. Such modifications include, for example, coding regions and functional fragments thereof, for heterologous, homologous, or both heterologous and homologous polypeptides for the referenced species. Additional modifications include, for example, non-coding regulatory regions in which the modifications alter expression of a gene or operon. Exemplary metabolic polypeptides include enzymes or proteins within a methane activation biosynthetic pathway.

As used herein, the term “host cell”, “host microbial organism”, and “host microorganism” are used interchangeably to refer to any archaeal, bacterial, or eukaryotic living cell into which a heterologous entity (e.g., a biomolecule such as a nucleic acid, protein, etc.) can be, or has been, inserted. The term also relates to the progeny of the original cell, which may not be completely identical in morphology or in genomic or total DNA complement to the original parent, due to natural, accidental, or deliberate mutation.

The terms “modified microorganism,” “recombinant microorganism”, and “recombinant host cell” are refer to a non-naturally occurring organism that is produced by methods such as inserting, expressing, or overexpressing endogenous polynucleotides; by expressing or overexpressing heterologous polynucleotides, such as those included in an integrated and/or episomal vector; by introducing a mutation into the microorganism; or by altering the expression of an endogenous gene. In embodiments relating to the introduction of a polynucleotide into a microorganism, the polynucleotide generally encodes a target enzyme involved in a metabolic pathway for producing a desired metabolite. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, e.g., a cell that has not been genetically modified. A wild-type microorganism can be genetically modified to express or overexpress a target enzyme. This microorganism can act as a parental microorganism in the generation of a microorganism modified to express or overexpress a target enzyme. In turn, the microorganism modified to express or overexpress one or more target enzymes can be modified to express or overexpress another target enzyme.

Accordingly, a “parental microorganism” functions as a reference cell for successive genetic modification events. Each modification event can be accomplished by introducing a nucleic acid molecule into the reference cell. The introduction facilitates the expression or overexpression of a target enzyme. It is understood that the term “facilitates” encompasses the activation of endogenous polynucleotides encoding a target enzyme through genetic modification of, e.g., a promoter sequence in a parental microorganism. It is further understood that the term “facilitates” encompasses the introduction of heterologous polynucleotides encoding a target enzyme in to a parental microorganism.

The term “mutation” as used herein indicates any modification of a nucleic acid that results in an altered nucleic acid, e.g., that produces an amino acid “substitution” in a polypeptide (e.g., thus producing a “mutant” polypeptide or “mutant” nucleic acid). Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are the results of artificial mutation pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (e.g., a metabolite) between the same substrate and metabolite end product.

The term “gene module” refers to a group, set, or collection of genes. In some embodiments, an operon comprises the genes of a gene module; in some embodiments, the genes of a gene module belong to the same metabolic pathway. In some embodiments, the genes of a gene module are coexpressed, e.g., the same set of transcription factors binds to the genes of the gene module to modulate expression of the genes of the gene module. In some embodiments, the genes of a gene module are provided together on a nucleic acid. In some embodiments, the genes are provided together on a nucleic acid in the same arrangement as found in nature and, in some embodiments, the genes of the gene module are provided on a nucleic acid in an arrangement that is not found in nature. In some embodiments, a gene module comprises a novel group, set, or collection of genes that are not normally present in the same pathway in nature. In some embodiments, a gene module comprises a novel group, set, or collection of genes that are not normally present in the same organism in nature.

The term “heterologous” as used herein with reference to molecules and in particular enzymes and polynucleotides, indicates molecules that are expressed in an organism other than the organism from which they originated or are found in nature, independently of the level of expression, which can be lower, equal, or higher than the level of expression of the molecule in the native microorganism.

Thus, the term “heterologous nucleic acid” refers to a nucleic acid wherein at least one of the following is true: (a) the nucleic acid is or comprises an exogenous nucleic acid; (b) the nucleic acid comprises a nucleotide sequence that is naturally found in (e.g., is “endogenous to”) a given host cell, but the nucleotide sequence is produced in an unnatural amount in the cell (e.g., greater or lesser than expected or greater or lesser than naturally found); (c) the nucleic acid comprises a nucleotide sequence that differs in sequence from an endogenous nucleotide sequence, but the nucleotide sequence encodes the same protein (having the same or substantially the same amino acid sequence) and is produced in an unnatural amount in the cell (e.g., greater or lesser than expected or greater or lesser than naturally found); and/or (d) the nucleic acid comprises two or more nucleotide sequences that are not found in the same relationship to each other in nature (e.g., two or more gene sequences are placed closer together and/or in a different order than naturally found in nature).

The term “exogenous” refers to something (e.g., a biomolecule (e.g., a nucleic acid (e.g., a RNA, a DNA), a protein, a lipid, a saccharide, etc.)) that is foreign to (e.g., not naturally found in) a given host cell. Thus, “exogenous” as it is used herein is intended to mean that the referenced molecule or the referenced activity is introduced into the host microorganism. The molecule or activity can be introduced, for example, by introduction of an encoding nucleic acid into the host genetic material such as by integration into a host chromosome or as non-chromosomal genetic material such as a plasmid. When the term “exogenous” is used in reference to expression of an encoding nucleic acid, it refers to introduction of the encoding nucleic acid in an expressible form into the host microorganism. When the term “exogenous” is used in reference to an enzymatic or protein activity, the term refers to an activity that is introduced into the host reference microorganism. When used in reference to a biosynthetic activity, the term refers to an activity that is introduced into the host microbial organism.

As used herein, the term “exogenous nucleic acid” refers to a nucleic acid that is exogenously introduced into a host cell, and hence is not normally or naturally found in and/or produced by a given cell in nature. Therefore, the term “exogenous” as it is used in reference to expression of an encoding nucleic acid refers to introduction of the encoding nucleic acid in an expressible form into the microbial host organism.

The source can be, for example, a homologous or heterologous encoding nucleic acid that expresses the referenced activity following introduction into the host microbial organism. Accordingly, exogenous expression of an encoding nucleic acid of the technology can utilize either or both a heterologous and/or a homologous encoding nucleic acid.

It is understood that when an exogenous nucleic acid is included in a microorganism that the exogenous nucleic acid refers to the referenced encoding nucleic acid or protein activity, as discussed above. It is also understood that such an exogenous nucleic acid can be introduced into the host microorganism on separate nucleic acid molecules, on a polycistronic nucleic acid molecule, on a single nucleic acid molecule encoding a fusion protein, or a combination thereof, and still be considered as more than one exogenous nucleic acid. For example, a microorganism can be modified to express two or more exogenous nucleic acids encoding an enzyme with a desired activity, e.g., methane oxidation, methyltransferase, sulfate reduction, hydrogenase, and other enzymes described herein (e.g., nucleic acids encoding methane oxidation, methyltransferase, sulfate reduction, hydrogenase, and other enzymes described herein). Where two or more exogenous nucleic acids are introduced into a host microorganism, it is understood that the two or more exogenous nucleic acids can be introduced as a single nucleic acid molecule (for example, on a single plasmid), can be introduced on separate plasmids, can be integrated into the host chromosome at a single site, or can be integrated into the host chromosome at multiple sites, and still be considered two exogenous nucleic acids. Similarly, it is understood that more than two exogenous nucleic acid molecules can be introduced into a host microorganism in any desired combination, for example, as a single nucleic acid molecule (for example, on a single plasmid), on separate plasmids, integrated into the host chromosome at a single site, or integrated into the host chromosome at multiple sites, and still be considered as two or more exogenous nucleic acids. Thus, the number of referenced exogenous nucleic acids or enzymatic activities refers to the number of encoding nucleic acids or the number of protein activities, not the number of separate nucleic acid molecules introduced into the host microorganism.

On the other hand, the term “native” or “endogenous” as used herein with reference to molecules, and in particular enzymes and polynucleotides, indicates molecules that are expressed in the organism in which they originated or are found in nature, independently of the level of expression, which can be lower, equal, or higher than the level of expression of the molecule in the native microorganism. It is understood that expression of native enzymes or polynucleotides may be modified in recombinant microorganisms.

Thus, the term “endogenous” or “native” refers to a referenced molecule or activity that is present in the referenced host microbial organism. Similarly, the term “endogenous” when used in reference to expression of an encoding nucleic acid refers to expression of an encoding nucleic acid contained within the host microbial organism.

As used herein, the term “chimeric” when used in reference to a nucleic acid refers to any nucleic acid that is not endogenous and that comprises sequences that are not found together in nature. For example, a chimeric nucleic acid may comprise regulatory sequences and coding sequences that are derived from different sources or regulatory sequences and coding sequences that are derived from the same source but arranged in a manner different than that found in nature.

The term “carbon source” generally refers to a substance suitable to be used as a source of carbon for prokaryotic or eukaryotic cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose. The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a modified microorganism as described herein.

As used herein, “methane” refers to the simplest alkane compound with the chemical formula CH₄. Methane is a colorless and odorless gas at room temperature and pressure. Sources of methane include natural sources, such as natural gas fields and biological synthesis via methanogenic microorganisms, and industrial or laboratory synthesis. Methane includes pure methane, substantially purified compositions, such as “pipeline quality natural gas” or “dry natural gas”, which is 95-98% percent methane, and unpurified compositions, such as “wet natural gas”, wherein other hydrocarbons have not yet been removed and methane comprises more than 60% of the composition.

As used herein, “natural gas liquids”, also known as “natural gas associated hydrocarbons” refers to the various hydrocarbons (e.g., ethane, propane, butane) that are separated from wet natural gas during processing to produce pipeline quality dry natural gas. “Partially separated derivative of wet natural gas” includes natural gas liquids.

As used herein, “reducing agent”, also known as “reductant,” “reducer,” or “reducing equivalent,” refers to an element or compound that donates an electron to another species.

As used herein, “methanol”, also known as “methyl alcohol” or “wood alcohol” refers to a colorless, water-soluble liquid having the chemical formula CH₃OH.

As used herein, a “metabolic modification” refers to a biochemical reaction that is altered from its naturally occurring state. In some embodiments, non-naturally occurring microorganisms have genetic modifications to nucleic acids encoding metabolic polypeptides or functional fragments thereof. In some embodiments, genetic modifications and/or metabolic modifications are described with reference to a suitable host organism such as, e.g., Methanococcus maripaludis and its corresponding metabolic reactions. In some embodiments, genetic modifications and/or metabolic modifications are described with reference to a suitable source organism for desired genetic material such as genes for a desired metabolic pathway.

The term “volumetric productivity” or “production rate” is defined as the amount of product formed per volume of medium per unit of time Volumetric productivity is reported in gram per liter per hour (g/L/h).

The term “specific productivity” is defined as the rate of formation of the product. To describe productivity as an inherent parameter of the microorganism or microorganism and not of the fermentation process, productivity is herein further defined as the specific productivity in gram product per unit of cells, typically measured spectroscopically as absorbance units at a wavelength of 600 nm (A₆₀₀, OD600, OD₆₀₀, or OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of butanol from glucose of 0.39 g/g would be expressed as 95% of theoretical or 95% theoretical yield.

The term “titre” or “titer” is defined as the strength of a solution or the concentration of a substance in solution. For example, the titre of a biofuel in a fermentation broth is described as g of biofuel in solution per liter of fermentation broth (g/L).

The term “total titer” is defined as the sum of all biofuel produced in a process, including but not limited to the biofuel in solution, the biofuel in gas phase, and any biofuel removed from the process and recovered relative to the initial volume in the process or the operating volume in the process.

A “facultative anaerobic organism” or a “facultative anaerobic microorganism” is defined as an organism that can grow in either the presence or in the absence of oxygen.

A “strictly anaerobic organism” or a “strictly anaerobic microorganism” is defined as an organism that cannot grow in the presence of oxygen and which does not survive exposure to any concentration of oxygen.

An “anaerobic organism” or an “anaerobic microorganism” is defined as an organism that cannot grow in the presence of oxygen.

“Aerobic conditions” are defined as conditions under which the oxygen concentration in the fermentation medium is sufficiently high for an aerobic or facultative anaerobic microorganism to use as a terminal electron acceptor.

In contrast, “anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. “Anaerobic conditions” are further defined as conditions under which no or small amounts of oxygen are added to the medium at rates of <3 mmol/L/h, preferably <2.5 mmol/L/h, more preferably <2 mmol/L/h and most preferably <1.5 mmol/L/h. “Anaerobic conditions” means in particular completely oxygen-free (e.g., 0 mmol/L/h oxygen) or with small amounts of oxygen added to the medium at rates of, e.g., <0.5 to <1 mmol/L/h.

Dissolved oxygen,” abbreviated as “DO” is expressed throughout as the percentage of saturating concentration of oxygen in water.

“Aerobic metabolism” refers to a biochemical process in which oxygen is used as a terminal electron acceptor to make energy, typically in the form of ATP, from carbohydrates. Aerobic metabolism occurs, e.g., via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways,” NAD(P)H donates its electrons to a molecule produced by the same metabolic pathway that produced the electrons carried in NAD(P)H. For example, in some fermentative pathways, NAD(P)H generated through glycolysis transfers its electrons to pyruvate, yielding lactate. Fermentative pathways are usually active under anaerobic conditions but may also occur under aerobic conditions, under conditions where NADH is not fully oxidized via the respiratory chain. For example, above certain glucose concentrations, some organisms produce large amounts of ethanol under aerobic conditions.

The term “fermentation product” means any main product plus its coupled product. A “coupled product” is produced as part of the stoichiometric conversion of the carbon source to the main fermentation product.

The term “byproduct” means an undesired product related to the production of a biofuel. Byproducts are generally disposed as waste, adding cost to a biofuel process.

The term “co-product” means a secondary or incidental product related to the production of biofuel. Co-products have potential commercial value that increases the overall value of biofuel production, and may be the deciding factor as to the viability of a particular biofuel production process.

As used herein, “cell-free fraction” refers to fractions (e.g., organelles, cytoplasm, cell extracts) that have been prepared from and/or separated from a cell. A cell-free fraction may be relatively unpurified (e.g., a cell lysate). Cell-free fractions also include relatively purified organelle fractions separated by differential centrifugation.

The term “polynucleotide” is used herein interchangeably with the term “nucleic acid” and refers to an organic polymer composed of two or more monomers including nucleotides, nucleosides, or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs, and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene”, refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (e.g., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

As used herein, the term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

As used herein, the term “regulatory element” refers to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection) is achieved by any one of a number of techniques including electroporation, chemical methods (e.g., calcium chloride method, polyethylene glycol method), and lipid mediated methods. Thus, the term “transformation” refers to a permanent or transient genetic change induced in a cell following introduction of new nucleic acid. Genetic change (“modification”) can be accomplished either by incorporation of the new DNA into the genome of the host cell, or by transient or stable maintenance of the new DNA as an episomal element. In eukaryotic cells, a permanent genetic change is generally achieved by introduction of the DNA into the genome of the cell. In prokaryotic cells, a permanent genetic change can be introduced into the chromosome or via extrachromosomal elements (“episomes”) such as plasmids and expression vectors, which may contain one or more selectable markers to aid in their maintenance in the recombinant host cell. Host microorganisms containing the transformed nucleic acid are referred to as “non-naturally occurring” or “recombinant” or “transformed” or “transgenic” microorganisms. Host microorganisms may be selected from, and the non-naturally occurring microorganisms generated in, any prokaryotic or eukaryotic microbial species from the domains of Archaea, Bacteria, or Eukarya. Accordingly, as used herein, the terms “transformed”, “stably transformed”, or “transgenic” with reference to a cell means the cell has a non-native (heterologous) nucleic acid sequence integrated into its genome or as an episomal plasmid that is maintained through multiple generations.

The term “viable” as used herein with reference to a genetically engineered microorganism indicates that the genetically engineered microorganism has a metabolic rate compatible with life and/or is able to reproduce. In some embodiments, a viable genetically engineered microorganism comprises a host microorganism that expresses the product of a heterologous nucleic acid and remains viable (e.g., expression of the heterologous nucleic acid in the host microorganism does not kill the host microorganism (e.g., does not kill the genetically engineered microorganism)). In some embodiments, a viable genetically engineered microorganism has normal and intact cells in a state of very low metabolic activity. In some embodiments, a viable genetically engineered microorganism does not reproduce, but has the ability to become reproduce and become culturable once resuscitated (e.g., under a favorable growth condition, e.g., in a culture medium comprising nutrients, carbon sources, salts, water, lipids, redox factors, etc.). In some particular embodiments, a viable genetically engineered microorganism is able to divide and form a colony on a nutrient medium appropriate for the growth of the genetically engineered microorganism and/or is able to increase the turbidity of liquid growth medium after inoculation with a preparation of the genetically engineered microorganism and incubation under appropriate conditions (e.g., appropriate temperature, agitation, and/or under aerobic and/or anaerobic atmosphere for an appropriate time) In some embodiments, molecular tools such as fluorescence in situ hybridization (FISH using “live/dead” staining) and flow cytometry are applied to estimate viable cells of the genetically engineered microorganism.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homolog” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural, or genomic similarities. Techniques are known by which homologs of an enzyme or gene can readily be cloned using genetic probes and PCR. The identity of cloned sequences as homologous can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, an “ortholog” is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species.

As used herein, a “paralog” is a homolog related by, for example, duplication followed by evolutionary divergence. Paralogs have similar or common, but not identical, functions. Paralogs can originate or derive from, for example, the same species or from a different species. Paralogs are proteins from the same species with significant sequence similarity to each other suggesting that they are homologous or related through co-evolution from a common ancestor.

As used herein, a “nonorthologous gene displacement” is a nonorthologous gene from one species that can substitute for a referenced gene function in a different species. Substitution includes, for example, being able to perform substantially the same or a similar function in the species of origin compared to the referenced function in the different species. Although, generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the technology having the capability to produce liquid fuels (e.g., alcohols such as, e.g., methanol, butanol, etc.) from alkanes such as methane, those skilled in the art will understand (e.g., with applying the teaching and guidance provided herein to a particular species) that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs, and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W, and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

As used herein, “gas phase bioreactor”, also known as a “gas phase reactor” refers to a manufactured or engineered device or system that supports a biologically active environment in which microorganisms or cell-free fractions derived from the microorganisms contact and catalyze a substrate. A biologically active environment may comprise microorganism growth or a chemical process carried by out the microorganisms (e.g., conversion of an alkane (e.g., methane) to an alcohol (e.g., methanol)) or catalytically active cell-free fractions derived from such microorganisms. Microorganisms or cell-free fractions derived thereof may be immobilized to a solid matrix within the reactor or to surfaces comprising the reactor itself.

As used herein, “fluidized bed reactor”, also known as “fluidized bed bioreactor,” refers to a bioreactor that is a combination of packed bed and stirred tank, continuous flow reactors. In a fluidized bed reactor, microorganisms or cell-free fractions derived thereof attached to particle bed carriers are suspended by upward passage of fluid (gas or liquid) such that the particle bed carriers freely circulate in the fluid. Fluidized bed reactors have excellent mass and heat transfer characteristics.

As used herein, “solid matrix” refers to a solid substance capable of having microorganisms or cell-free fractions derived from the microorganisms temporarily or permanently attached on, within, or behind the solid matrix. Immobilized microorganisms may grow on the surface of the solid matrix (e.g., as a biofilm). Solid matrix supports may be in a variety of shapes, such as sheets, rings, beads, and may comprise a number of materials, including polypropylene, sand, granular activated carbon, diatomaceous earth, and ceramics.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### DESCRIPTION

In some embodiments, the present technology provides a non-naturally occurring microbial organism that comprises a biosynthetic pathway and/or activity for activating methane. The non-naturally occurring microbial organism includes at least one exogenous nucleic acid encoding a methane activation pathway enzyme expressed in a sufficient amount to activate methane. In some embodiments, a methane activation pathway includes a methyl reductase such as a coenzyme M reductase (MCR) enzyme. Although the disclosure herein refers to certain illustrated embodiments, it is to be understood that these embodiments are presented by way of example and not by way of limitation.

### Methane

Methane is the simplest organic compound and has the highest energy content of any carbon-based fuel (e.g., producing approximately 55.7 kJ/g). Methane is widely mined and used as a fuel for heating and cooking, and is used by the chemical industry to produce synthesis gas (“syngas”, a mixture of CO and H₂), to generate electricity, and to serve as a vehicle fuel in the form of compressed or liquid natural gas. Methane utilization has environmental ramifications because it is a potent greenhouse gas whose levels have doubled over the past two centuries.

A class of microbes called methanogens generates nearly all of the methane on earth and produces it at a level of 1 billion tons per year. The methane produced is not only used as a source of energy for human consumption as described above, but methanotrophic microbes capture emitted methane and oxidize it as a source of cell carbon and energy as a key part of the global carbon cycle. In fact, much of the methane produced by methanogens is recaptured and used as an energy source by methanotrophic microbes. These methanotrophs include aerobic bacteria that oxidize methane and organisms that oxidize methane anaerobically. In fact, large amounts (0.3 billion tons per year) of methane are oxidized to CO₂ in marine sediments and shallow lagoon settings at a depth interval known as the sulfate-methane transition zone. These environments comprise anaerobic methanotrophic archaea (e.g., ANME organisms, e.g., of groups ANME-1, ANME-2, or ANME-3, in addition to any ANME or similar organisms yet to be discovered) and sulfate-reducing or nitrate-reducing bacteria.

Fluorescence in situ hybridization studies and phylogenetic analyses reveal that methane-oxidizing archaea and sulfate-reducing bacteria are closely associated in the sulfate-methane transition zone (see, e.g., Orphan et al (2001) Science 293: 484-7; Orphan et al (2002) Proc Natl Acad Sci USA 99: 7663-8), thus indicating that methane oxidation involves a consortium of microbes in which one population catalyzes anaerobic oxidation of methane and another oxidizes a substrate such as sulfate.

### Pathways

Accordingly, provided herein is technology related to a novel and transformational bioengineering technology for the biological synthesis of liquid fuels (e.g., methanol) from an alkane (e.g., methane). In some embodiments, this technology provides a methanogenic host bacterium (e.g., Methanococcus maripaludis) comprising a heterologous metabolic pathway (e.g., a gene module comprising one or more genes) to provide a non-natural organism derived from M. maripaludis that converts an alkane (e.g., methane) to a fuel (e.g., an alcohol such as, e.g., methanol). Anaerobic methane oxidation is highly energy efficient. For example, the energy efficiency associated with a genetically engineered anaerobic methane-to-butanol conversion has been estimated to be approximately 77%. This exceptional product yield is due to highly efficient substrate conversion by anaerobic pathways to compensate for the low yield of ATP and marginally favorable thermodynamics (e.g., −21 kJ/mol) for anaerobic oxidation of methane.

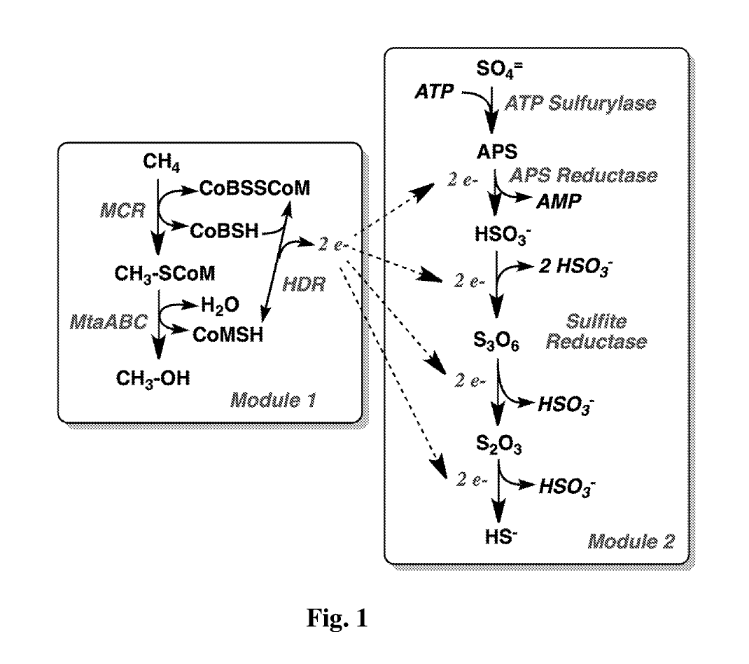

An exemplary pathway (FIG. 1, Module 1) comprises a methyl-coenzyme M reductase (MCR) that converts methane (CH₄) and a heterodisulfide of coenzyme M and coenzyme B (CoBS-SCoM) to methyl-coenzyme M (CH₃-SCoM) and coenzyme B (CoBSH):

CH₄+CoBS-SCoM→CH₃-SCoM+CoBSH

This first step provides an activated reaction intermediate, e.g., “activated” methane in the form of methyl-coenzyme M (CH₃-SCoM), for further reaction. Coenzyme M is 2-mercaptoethanesulfonate (HSCH₂CH₂SO₃⁻), which participates in methyl transfer reactions in the metabolism of methanogens and methanotrophs. Coenzyme B is 7-mercaptoheptanoylthreoninephosphate, which participates in certain oxidation/reduction reactions in methanogens and methanotrophs.

For example, in a subsequent step, a methanol:coenzyme M methyltransferase enzyme (MtaABC) reacts the methyl-coenzyme M intermediate with water (H₂O) to form coenzyme M (CoMSH) and methanol (CH₃OH) (FIG. 1, Module 1):

CH₃-SCoM+H₂O→CoMSH+CH₃OH

MtaABC comprises MtaA, MtaB, and MtaC polypeptides, e.g., as the expression products of an mtaABC operon or a gene module comprising the mtaA, mtaB, and mtaC genes. In an exemplary embodiment, mtaA, mtaB, and mtaC genes cloned from Methanosarcina barkeri are heterologously expressed in the host microorganism to convert methyl-SCoM to methanol (and regenerate the coenzyme M (HSCoM) and coenzyme B (HSCoB) cofactors). The Methanosarcina barkeri mtaA, mtaB, and mtaC genes and the Methanosarcina barkeri methyltransferase expressed from the mtaA, mtaB, and mtaC genes are both extensively characterized.

Accordingly, embodiments of the technology are related to an integrated system comprising a methane-oxidizing pathway (e.g., comprising methyl-coenzyme M reductase) and a methanol-producing pathway (e.g., comprising methanol:coenzyme M methyltransferase) that provides a genetically engineered metabolic pathway for converting methane to methanol. Accordingly, in an exemplary embodiment, the conversion of methane to methanol is described by:

CH₄+H₂O→CH₃OH+H₂ΔG°′=112.5 kJ/mol

In nature, anaerobic oxidation of methane appears to involve a syntrophic system in which the endergonic process of anaerobic oxidation of methane is thermodynamically coupled to and driven by the exergonic dissimilatory reduction of sulfate by a sulfate reducing organism (see, e.g., Orphan et al (2001) Science 293: 484-7; Orphan et al (2002) Proc Natl Acad Sci USA 99: 7663-8). Further, organisms related to ANME-2 have been shown to couple anaerobic oxidation of methane to the microbial reduction of nitrate to dinitrogen gas, yielding a significantly more favorable energy balance (see, e.g., Raghoebarsing et al. (2006) Nature 440: 918-21).

As such, in some embodiments the methanol producing pathway (e.g., comprising methane-oxidizing MCR and the methanol-producing Mta described above) is coupled to an exergonic electron-accepting pathway that will render the overall process exergonic. However, in contrast to the natural system comprising a consortium of independent organisms, the technology provided herein provides an integrated process of methane oxidation wherein the oxidation of methane and the coupled exergonic process occurs in a single genetically engineered organism.

For instance, in some embodiments, the technology comprises coupling the conversion of an alkane (e.g., methane) to a fuel (e.g., an alcohol such as methanol) to another biochemical pathway in a genetically engineered microorganism such that the two coupled pathways provide a thermodynamically favorable system. For example, in some embodiments, the conversion of methane to methanol (FIG. 1, Module 1) is coupled to the conversion of sulfate to sulfide (FIG. 1, Module 2). In some embodiments, a heterodisulfide reductase enzyme (HDR) reacts coenzyme M and coenzyme B to form the heterodisulfide of coenzyme M and coenzyme B, which provides electrons for the conversion (e.g., reduction) of sulfate to sulfide in the coupled system (FIG. 1, Module 1 and Module 2). In some embodiments, a pathway for the reduction of sulfate to sulfide comprises enzymes or activities that are an ATP sulfurylase, an APS reductase, and/or a sulfite reductase.

Some embodiments provide enzymes from the canonical dissimilatory sulfate reduction pathways. In particular embodiments, the technology provides enzymes and genes encoding enzymes that are from an organism that is part of a natural methane-oxidizing consortium (see, e.g., Basen et al (2011) Environ Microbiol 13: 1370-9). In some embodiments, the technology provides enzymes and genes encoding enzymes that are related to the production of zero-valent sulfur by a non-canonical pathway of sulfate reduction.

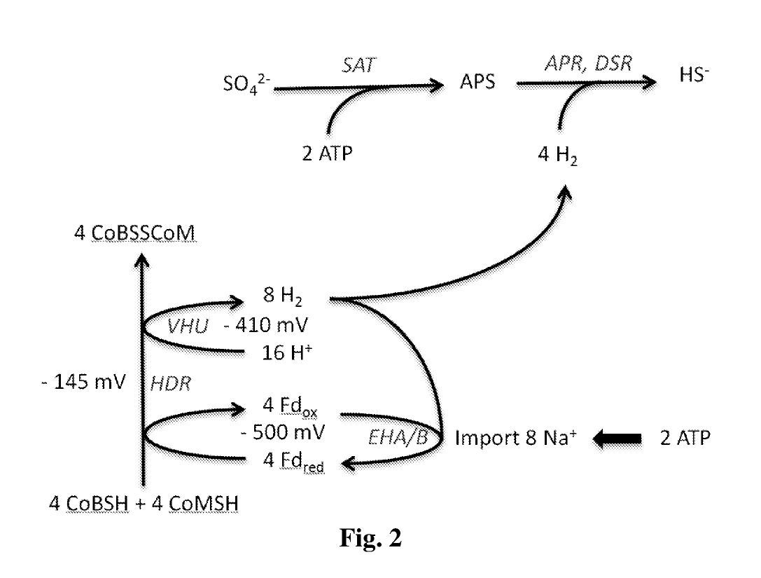

Embodiments of the technology provide a system for methanol production in which the methane oxidation pathway is coupled to a canonical sulfate reduction pathway (see, e.g., FIG. 1). In some embodiments, the methane oxidation pathway is coupled to a sulfate reduction pathway comprising a heterodisulfide reductase (Hdr) that converts (e.g., by oxidation) HSCoM and HSCoB to CoBS-SCoM. The production of CoBS-SCoM by Hdr produces reduced ferredoxin (Fd) and H₂ via the hydrogenase Vhu (FIG. 2). The reduced ferredoxin is then used by the energy-converting hydrogenase Eha to produce H₂ in an energy-conserving reaction that produces a chemiosmotic membrane potential. The H₂ produced by the pathway is used to reduce sulfate (FIG. 2). Some embodiments provide the metabolic pathway under thermodynamically feasible conditions, e.g., at the ambient low partial pressure of H₂. Furthermore, embodiments of the pathway produce ATP; in some embodiments the ATP is produced chemiosmotically, which activates sulfate and supports growth of an organism comprising the pathway.

This mechanism for the generation of a chemiosmotic potential and ATP production is proposed to occur naturally in organisms such as those of the class Methanobacteria (e.g., in the order Methanobacteriales, family Methanobacteriaceae, and/or the genus Methanosphaera, e.g., Methanosphaera stadtmanae) (see, e.g., Thauer et al (2008) Nat Rev Microbiol 6: 579-91).

A similar pathway is also present in M. maripaludis (see, e.g., Costa et al (2013) MBio 4). Accordingly, in some embodiments, expression of the M. maripaludis gene for glyceraldehyde-3-phosphate:ferredoxin oxidoreductase (GAPOR) is induced, which produces a metabolic cycle in which GAPOR is coupled with steps in gluconeogenesis. This engineered metabolic coupling is reversible: in one direction, the coupled pathway provides electron flow from NADPH to ferredoxin and produces hydrolysis of ATP. In the other direction, reduced ferredoxin transfers electrons to NADP (e.g., reduces NADP) and produces ATP. Subsequently, NADPH reduces F₄₂₀, from which H₂ is produced.

In some embodiments, a non-natural organism comprises a heterologous pathway for sulfate activation and reduction. In some embodiments, the non-natural organism is derived from M. maripaludis, which is an organism that comprises enzymes and pathways for coupling methane oxidation to sulfate reduction. In some embodiments, a genetically engineered M. maripaludis comprises enzymes and pathways for coupling methane oxidation to sulfate reduction and further comprises heterologous genes, pathways, and proteins for sulfate activation and reduction.

In exemplary embodiments, a host organism (e.g., M. maripaludis) comprises a sulfate-reducing pathway (see e.g., FIGS. 1 and 2). In some embodiments, a host organism (e.g., M. maripaludis) expresses cloned genes that encode one or more of an ATP sulfurylase (Sat), an APS reductase (Apr), and a sulfite reductase (Dsr) (see, e.g., FIGS. 1 and 2). In some embodiments, the adenosine 5′-phosphosulfate reductase (e.g., Apr) reduces adenosine 5′-phosphosulfate (APS) to sulfite in the biological process of dissimilatory sulfate reduction.

In some embodiments, a QmoABC complex is involved in sulfate reduction and interacts with Apr in electron transfer. The QmoABC complex has one membrane (QmoC) and two cytoplasmic subunits (QmoAB); QmoC comprises two heme b cofactors that are reduced by quinols to provide electron flow between quinone and other components in the cytoplasm.

In some embodiments, sulfate reduction is an intracellular process related to active transport of sulfate and its activation by reaction with ATP to form adenosine 5′-phosphosulfate (APS). The two terminal reductases, APS reductase (Apr) and dissimilatory sulfite reductase (Dsr), transfer electrons to a QmoABC complex, which provides for electron flow between a quinone (e.g., a menaquinone) and APS reduction or oxidation by Apr. In some embodiments, a DsrMKJOP is involved in electron transfer (e.g., with the sulfite reductase Dsr.

In some embodiments, the cloned genes are from a source organism that is a sulfate reducer (e.g., an organism from the genus Desulfovibrio, e.g., D. desulfuricans or D. vulgaris; genus Chlorobium, e.g., C. tepidum;). While some methanogens are sensitive to sulfite, the sulfite reductase activity provides resistance to sulfite produced by the pathway (see, e.g., Johnson & Mukhopadhyay (2008) Appl Environ Microbiol 74: 3591-5).

In some organisms, electrons are provided to the Apr and Dsr enzymes by reverse electron bifurcation (electron confurcation) through an enzyme (e.g., QmoABC) present in sulfate reducers that is homologous to Hdr (see, e.g., Grein et al (2013) Biochim Biophys Acta 1827: 145-60). In some embodiments electron flow to sulfite occurs through a complex of Dsr proteins and Hdr-like proteins. Thus, in some embodiments a genetically engineered organism comprises heterologous pathways comprising a QmoABC complex and other electron providers.

Further, in some embodiments electrons are provided to Qmo and/or to the Dsr complex by genetic engineering that manipulates the electron flow pathways in the host organism (e.g., M. maripaludis) as necessary. For example, M. maripaludis comprises pathways for electron flow between H₂, Fd, NAD(P), and F₄₂₀. Accordingly, M. maripaludis comprises a variety of electron donors that are manipulable by genetic engineering to provide electron flow to pathways as appropriate (see, e.g., Costa et al (2013) MBio 4).

In some embodiments, a genetically engineered organism comprises heterologous pathways comprising enzymes for sulfate reduction from the sulfate-reducing partner of the ANME consortium. For example, sulfate reducers in the ANME consortium may use HSCoM and HSCoB to reduce APS or sulfite, or may reduce sulfate without activation to APS using electrons from the low-potential Fd (see, e.g., Thauer et al (2008) Nat Rev Microbiol 6: 579-91). In some embodiments, a genetically engineered organism comprises heterologous pathways for sulfite reduction by an assimilatory sulfite reductase enzyme. Such an enzyme is present naturally in methanogens. This enzyme, Fsr, is an F₄₂₀-dependent sulfite reductase that is present in Methanocaldococcus jannaschii. The M. jannaschii Fsr was expressed in M. maripaludis and conferred resistance to sulfite by converting it to sulfide (see, e.g., Johnson & Mukhopadhyay (2008) Appl Environ Microbiol 74: 3591-5). Fsr is also present in the M. maripaludis relatives Methanothermococcus thermolithotrophicus and the mesophilic Methanococcus aeolicus (see, e.g., Susanti & Mukhopadhyay (2012) PLoS One 7, e45313), suggesting the gene is available from several source organisms.

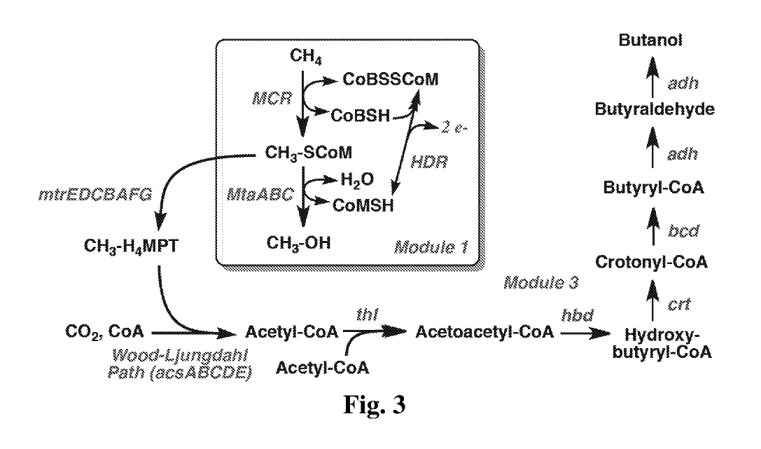

Further, some embodiments provide for the conversion of one alcohol to another alcohol, e.g., the conversion of methanol to butanol. Particular embodiments provide cloned genes for the conversion of one alcohol to another alcohol, e.g., the conversion of methanol to butanol. An exemplary pathway comprises conversion of methanol and CO₂ to acetyl-CoA through the Wood-Ljungdahl pathway, which is present in host organisms such as M. maripaludis. Then, acetyl-CoA is converted to butanol through intermediates in a pathway that converts acetoacetyl-CoA to hydroxybutyryl-CoA, followed by conversion of hydroxybutyryl-CoA to butenyl (crotonyl)-CoA, and finally by conversion of butenyl (crotonyl)-CoA to butyryl-CoA (see, e.g., FIG. 3). This pathway is present in source organisms such as solvent-producing clostridia (e.g., Clostridium carboxidivorans) that comprise the Wood-Ljungdahl pathway and a metabolic pathway for butanol production (see, e.g., Bruant et al (2010) PLoS One 5: e13033). Furthermore, this pathway also exists in C. acetobutylicum and has been successfully engineered into E. coli (see, e.g., Atsumi et al. (2008) Metab Eng 10: 305-11) and S. cerevisiae (see, e.g., Steen et al. (2008) Microb Cell Fact 7: 36). In C. carboxidivorans and other solventogenic clostridia, the genes involved in the conversion of acetyl-CoA to butyryl-CoA are in a gene cluster on the chromosome. In some embodiments, the transfer of a methyl group from methyl-coenzyme M to a methyltetrahydromethanopterin (CH₃-H₄MPT) is catalyzed by a methyltransferase such as a methyltransferase complex (e.g., a multi-subunit methyltransferase such as mtrA-H or mtrEDCBAFG (see, e.g., FIG. 3)). Some methyltransferases comprise a 5-oxybenzimidazolyl cobalamin cofactor that participates in the methyl transfer. Some methyltransferases are a Na⁺-translocating methyltransferase.

In some embodiments, a M. maripaludis strain comprising a sulfate reduction pathway comprises a sequence of electron transfer components that provides for the conversion of methane and/or methanol to butanol.

Some embodiments provide a non-naturally occurring organism in which a methylene-H₄MPT (methylene-tetrahydro-methanopterin) reductase gene (mer) has been inactivated, e.g., by deletion, knock-out, knock-in, etc. Some embodiments provide a non-naturally occurring organism in which a methylene-H₄MPT reductase gene (mer) is modulatable (e.g., its activity can be increased, decreased, induced, inhibited, enhanced, etc.). The mer gene is involved in oxidation of methyl-SCoM to CO₂. Thus, an inactive mer increases metabolic flux to methanol in the absence of the methanol-to-butanol pathway and an active mer increases metabolic flux to butanol. Accordingly, embodiments provide for the control of metabolic flux and production of methanol or butanol by control of the activity of the mer gene. In particular embodiments comprising a sulfate reduction path, metabolic flux of methane to methanol (or butanol) is efficient in the mer⁻ strains.

Accordingly, in some embodiments a non-natural organism comprising a heterologous pathway (e.g., a gene module comprising one or more genes) for the conversion of an alkane (e.g., methane) to an alcohol (e.g., methanol) also comprises a pathway (e.g., a gene module comprising one or more genes) for the conversion of sulfate to sulfide. In some further embodiments, the technology further provides for the subsequent conversion of one alcohol (e.g., methanol) to another alcohol (e.g., butanol), e.g., by a pathway (e.g., a gene module comprising one or more genes) for the conversion of one alcohol (e.g., methanol) to another alcohol (e.g., butanol). In some embodiments, the genetically engineered microorganism comprises the pathway for the conversion of one alcohol (e.g., methanol) to another alcohol (e.g., butanol) in addition to the pathways for conversion of an alkane (e.g., methane) to an alcohol (e.g., methanol) and conversion of sulfate to sulfide.

Accordingly, in some embodiments are provided a technology for converting an alkane to an alcohol, such as converting methane to methanol and, in some embodiments, further to butanol. In some embodiments, a pathway for converting an alkane (e.g., methane) to an alcohol (e.g., methanol, butanol) is coupled to another pathway such that conversion of the alkane in the coupled pathway is thermodynamically favorable.

The technology described herein provides a novel bioengineering approach to convert methane to liquid fuels. The technology comprises use of a genetically engineered microorganism to catalyze the anaerobic oxidation of methane to methanol. As described above, embodiments provide a thermodynamically favorable pathway system for the anaerobic oxidation of methane to methanol by coupling the conversion to another metabolic pathway. Accordingly, in some embodiments, the genetically engineered microorganism comprises a pathway (e.g., a gene module comprising one or more genes), providing sulfate reduction. Further, in some embodiments the genetically engineered microorganism comprises a pathway (e.g., a gene module comprising one or more genes) providing for the conversion of methanol to butanol.

### Methyl Reductase

A key enzymatic catalyst for this high level of substrate conversion by anaerobic oxidation of methane is methyl-coenzyme M reductase (“MCR”, EC 2.8.4.1). MCR is a nickel-containing enzyme of approximately 300 kDa that is unique to methanogens and anaerobic methane oxidizers. MCR catalyzes the rate-limiting and final step of methane production by methanogens:

CH₃-SCoM+CoBSH→CH₄+CoBS-SCoM ΔG°′=−30 kJ/mol

where CH₃-SCoM is methyl-coenzyme M, CoBSH is coenzyme B, CH₄ is methane, and CoBS-SCoM is a heterodisulfide of coenzyme M and coenzyme B. MCR is a homodimer, with each monomer comprising an α subunit, β subunit, and γ subunit, which are also called McrA, McrB and McrG, respectively. MCR has two active sites, each occupied by the nickel-containing F₄₃₀ cofactor. The MCR reaction is tightly coupled, with methyl-SCoM being converted into methane (in the methane synthesis direction) with 100% carbon conversion efficiency.

While production of methane by MCR in methanogens has long been studied, a recent surprising finding is that methyl-coenzyme M reductase also catalyzes the oxidation of methane (“AOM”) in the reverse process, e.g., the activation of methane by the production of methyl-coenzyme M:

CH₄+CoBS-SCoM→CH₃-SCoM+CoBSH

As an example, the ANME MCR involved in anaerobic oxidation of methane involves the same pathway components, e.g., the sulfhydryl coenzymes HSCoM and HSCoB, and the heterodisulfide CoMS-SCoB, as are used by methanogens in the reverse process for methane production (see, e.g., Shima et al. (2012) Nature 481: 98-101). Accordingly, based on the pathway scheme the products of methane oxidation are methyl-SCoM and HSCoB (FIG. 1). Based on the principle of microreversibility and the 100% carbon conversion efficiency for the conversion of methyl-SCoM to methane, the conversion of methane to methyl-coenzyme M should also have a 100% carbon conversion efficiency. Further, the methanol methyltransferase reaction that catalyzes the conversion of methyl-SCoM to methanol is also tightly coupled and thus, in addition to the favorable conversion efficiency, the process is expected to convert methane to methanol quantitatively.

Two forms of MCR exist. MCR-I (e.g., encoded by an operon comprising mcrB, mcrD, mcrC, mcrG, and mcrA) is present in all methanogens. The isoenzyme MCR-II (e.g., encoded by an operon comprising mrtB, mrtD, mrtG, and mrtA) is present in some members of the Methanococci and Methanobacteria. In addition to the genes encoding the MCR α subunit, β subunit, and γ subunit, the MCR operon comprises additional genes encoding proteins (e.g., McrC and McrD in MCR-I; MrtD in MCR-II) whose function is unknown. These additional proteins may be involved in post-translational modification of the a subunit, which contains 4 methylated residues and one thiolated glycine.

The mcrA gene produces the McrA subunit (UniProt 027232), which has a molecular weight of approximately 60 kDa; The mcrB gene produces the McrB subunit (UniProt 027236), which has a molecular weight of approximately 47 kDa; The mcrG gene produces the McrG subunit (UniProt 027233), which has a molecular weight of approximately 29 kDa.

Under conditions of limited H₂ and CO₂, MCR I predominates; when the concentrations of H₂ and CO₂ are not growth-limiting, MCR II predominates. The reaction is, for both enzymes, via ternary complex and takes place under anaerobic conditions.

Purified MCR protein contains 2 moles each of coenzyme F₄₃₀, coenzyme M, and coenzyme B. The purified enzyme is active when the Ni atom at the center of the coenzyme F₄₃₀ cofactor is reduced to the Ni(I) oxidation state, e.g., by treatment with titanium (III) citrate. N-8-mercaptooctanoylthreonine phosphate, 6-mercaptohexanoylthreonine phosphate, 2-azidoethanesulfonate, 4-bromobutanesulfonate, 3-bromopropanesulfonat, and 2-bromoethanesulfonate are competitive inhibitors of the enzyme (e.g., competitors of coenzyme B and/or coenzyme M). Such inhibitors are useful in the study and characterization of MCR.

In some embodiments of the technology, a vector is used comprising an mcr operon or comprising one or more mcr genes (e.g., comprising mcrB, mcrD, mcrC, mcrG, and/or mcrA). In some embodiments, the mcr operon or mcr genes are cloned from an ANME organism (e.g., from ANME-1, ANME-2, or ANME-3) or from Methanothermobacter marburgensis. Some embodiments provide a genetically engineered microorganism comprising a genome comprising one or more mcr genes (e.g., comprising mcrB, mcrD, mcrC, mcrG, and/or mcrA). In some embodiments the one or more mcr genes (e.g., comprising mcrB, mcrD, mcrC, mcrG, and/or mcrA) is/are integrated into the chromosome of the genetically engineered microorganism. In some embodiments, the one or more mcr genes (e.g., comprising mcrB, mcrD, mcrC, mcrG, and/or mcrA) is/are present on an episomal (e.g., extrachromosomal) genetic component (e.g., a plasmid vector).

With the complete genome sequence available for thousands of microorganisms, the identification of genes encoding an enzyme with methyl coenzyme M reductase activity in related or distant species, including for example, homologs, orthologs, paralogs, etc., is contemplated. Accordingly, exogenous nucleic acids encoding an enzyme with methyl coenzyme M reductase activity described herein with reference to particular nucleic acids from a particular microorganism can readily include other nucleic acids encoding an enzyme with coenzyme M reductase activity from other microorganisms.

Functional constraints on the catalytic activity of MCR have resulted in a high degree of amino acid sequence conservation. This conserved primary structure has been used to develop degenerate PCR primers for recovering naturally occurring mcrA fragments from a variety of environments. For example, PCR techniques, primers, and alignments are available for identifying mcrA genes in all five orders of Archaea. See, e.g., Luton, et al (2002), “The mcrA gene as an alternative to 16S rRNA in the phylogenetic analysis of methanogen populations in landfill”, Microbiology 148: 3521; see Hallam et al (2003), “Identification of Methyl Coenzyme M Reductase A (mcrA) Genes Associated with Methane-Oxidizing Archaea”, Appl. Environ. Microbiol. 69: 5483; Hallam et al. (2004) “Reverse Methanogenesis: Testing the hypothesis with environmental genomics”, Science 305: 1457. For example, in some embodiments MCR genes (e.g., mcrB, mcrD, mcrC, mcrG, and/or mcrA; mrtB, mrtD, mrtG, and/or mrtA) are identified and/or cloned using amplification of nucleic acids (e.g., from an environmental sample, from a culture, co-culture, etc.). In some embodiments, primers for amplification of MCR genes are provided by Hales, et al (1996) “Isolation and identification of methanogen-specific DNA from blanket bog peat by PCR amplification and sequence analysis”, Appl Environ Microbiol. 62: 668-675; Hallam et al (2003), “Identification of Methyl Coenzyme M Reductase A (mcrA) Genes Associated with Methane-Oxidizing Archaea”, Appl. Environ. Microbiol. 69: 5483; Hallam et al. (2004) “Reverse Methanogenesis: Testing the hypothesis with environmental genomics”, Science 305: 1457.

In some embodiments, MCR genes have or are derived from sequences provided by any one or more of NCBI accession numbers AY714814 to AY714873, AY324362 to AY324373, and/or AY327048 to AY327049 (e.g., comprising complete mcrA operons), incorporated herein by reference.

In some embodiments, the methylreductase is a M. maripaludis methylreductase that is the native M. maripaludis methylreductase (e.g., it is not a heterologous methylreductase). In some embodiments, the methylreductase is the native M. maripaludis methylreductase comprising one or more mutations in the gene and/or one or more substitutions in the protein to provide a genetically engineered methylreductase with improved characteristics such as higher efficiency of product production, increased stability, etc.

In some embodiments, genes described herein are produced by direct synthesis.

Polypeptide sequences and encoding nucleic acids for proteins, protein domains, and fragments thereof described herein, such as an enzyme with coenzyme M reductase activity, may include natural and recombinantly engineered variants. A nucleic acid variant refers to a nucleic acid that may contain one or more substitutions, additions, deletions, insertions, or may be or comprise fragment(s) of a reference nucleic acid. A reference nucleic acid refers to a selected wild-type or parent nucleic acid encoding an enzyme with coenzyme M reductase activity. A variant nucleic acid may have 40%, 45%, 50%, 55%, 60%, 65%, 70%, 75%, 80%, 85%, 90%, 95%, 96%, 97%, 98%, or 99% sequence identity to a reference nucleic acid, as long as the variant nucleic acid encodes a polypeptide that can still perform its requisite function or biological activity (e.g., producing alcohol from alkane). A variant polypeptide may have 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a reference protein, as long as the variant polypeptide can still perform its requisite function or biological activity (e.g., producing alcohol from alkane). In certain embodiments, an enzyme with coenzyme M reductase activity that is introduced into non-naturally occurring microorganisms according to any of the embodiments disclosed herein comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence encoded by a nucleic acid sequence provided by any one or more of accession numbers AY327049, AY327048, FP565147, JMIY01000002, JX465656, GU182110, HM746616, HM746615, HM746614, HM746613, HM746612, HM746611, HM746610, HM746609, HM746608, HM746607, HM746606, HM746605, HM746604, HM746603, HM746602, JMIY01000005, HQ651870, HQ651869, DQ521864, DQ521863, DQ521862, DQ521861, DQ521860, DQ521859, DQ521858, DQ521857, AB560814, AB560813, AB560812, AB560811, AB560810, AB525499, AB525498, AB525497, AB525496, AB525495, AB525494, AB525493, AB525492, HQ651881, HQ651880, HQ651879, HQ651878, HQ651877, HQ651876, HQ651875, HQ651874, HQ651873, HQ651872, HQ651871, HQ651868, or HQ651867. These accession numbers provide the nucleotide sequences of full or partial mcr genes, other genes described herein, and/or full or partial genome or putative genome sequences of organisms having one or more mcr genes and/or other genes described herein.

Variants may have improved function and biological activity (e.g., higher enzymatic activity or improved specificity for substrate) than the parent (or wild-type) protein. Due to redundancy in the genetic code, nucleic acid variants may or may not affect amino acid sequence. A nucleic acid variant may also encode an amino acid sequence comprising one or more conservative substitutions compared to a reference amino acid sequence. A conservative substitution may occur naturally in the polypeptide (e.g., naturally occurring genetic variants) or may be introduced when the polypeptide is recombinantly produced. A conservative substitution is where one amino acid is substituted for another amino acid that has similar properties, such that one skilled in the art would expect that the secondary structure and hydropathic nature of the polypeptide to be substantially unchanged. Amino acid substitutions may generally be made on the basis of similarity in polarity, charge, solubility, hydrophobicity, and/or the amphipathic nature of the residues, and is known in the art. Amino acid substitutions, deletions, and additions may be introduced into a polypeptide using well-known and routinely practiced mutagenesis methods (see, e.g., Sambrook et al. Molecular Cloning: A Laboratory Manual, 3d ed., Cold Spring Harbor Laboratory Press, N Y 2001).

Oligonucleotide-directed site-specific (or segment specific) mutagenesis procedures may be employed to provide an altered polynucleotide that has particular codons altered according to the substitution, deletion, or insertion desired. Deletion or truncation variants of proteins may also be constructed by using convenient restriction endonuclease sites adjacent to the desired deletion. Alternatively, random mutagenesis techniques, such as alanine scanning mutagenesis, error prone polymerase chain reaction mutagenesis, and oligonucleotide-directed mutagenesis may be used to prepare polypeptide variants (see, e.g., Sambrook et al, supra). Nucleic acids encoding an enzyme with coenzyme M reductase activity may be combined with other nucleic acid sequences, such as promoters, polyadenylation signals, restriction enzyme sites, multiple cloning sites, other coding segments, and the like.

Differences between a wild-type (or parent) nucleic acid or polypeptide and the variant thereof, may be determined by methods routinely practiced in the art to determine identity, which are designed to give the greatest match between the sequences tested. Methods to determine sequence identity can be applied from publicly available computer programs. Computer program methods to determine identity between two sequences include, for example, BLASTP, BLASTN (Altschul, S. F. et al, J. Mol. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al, NCBI NLM NIH Bethesda, Md.

Assays for determining whether a polypeptide variant folds into a conformation comparable to the non-variant polypeptide or fragment include, for example, the ability of the protein to react with mono- or polyclonal antibodies that are specific for native or unfolded epitopes, the retention of ligand-binding functions, the retention of enzymatic activity (if applicable), and the sensitivity or resistance of the mutant protein to digestion with proteases (see Sambrook et al, supra). Polypeptides, variants and fragments thereof, can be prepared without altering a biological activity of the resulting protein molecule (e.g., without altering one or more functional activities in a statistically significant or biologically significant manner). For example, such substitutions are generally made by interchanging an amino acid with another amino acid that is included within the same group, such as the group of polar residues, charged residues, hydrophobic residues, and/or small residues, and the like. The effect of any amino acid substitution may be determined empirically merely by testing the resulting modified protein for the ability to function in a biological assay, or to bind to a cognate ligand or target molecule.

### Codon Optimization

Expression of recombinant proteins is often difficult outside their original host. For example, variation in codon usage bias has been observed across different species of bacteria (Sharp et al, 2005, Nucl. Acids. Res. 33: 1 141-1 153). Over-expression of recombinant proteins even within their native host may also be difficult. In certain embodiments of the technology, nucleic acids (e.g., a nucleic acid encoding an enzyme with coenzyme M reductase activity) that are to be introduced into microorganisms according to any of the embodiments disclosed herein may undergo codon optimization to enhance protein expression. Codon optimization refers to alteration of codons in genes or coding regions of nucleic acids for transformation of an organism to reflect the typical codon usage of the host organism without altering the polypeptide for which the DNA encodes. Codon optimization methods for optimum gene expression in heterologous organisms are known in the art and have been previously described (see, e.g., Welch et al (2009), PLoS One 4: e7002; Gustafsson et al (2004), Trends Biotechnol. 22: 346-353; Wu et al (2007), Nucl. Acids Res. 35: D76-79; Villalobos et al (2006), BMC Bioinformatics 7: 285; U.S. Pat. App. Pub. No. 2011/0111413; and U.S. Pat. App. Pub. No. 2008/0292918).

A discussion of codon usage in M. maripaludis in particular is provided by Emery and Sharp (2010) “Impact of translational selection on codon usage bias in the archaeon Methanococcus maripaludis”, Biol. Lett. rsbl20100620 (doi: 10.1098/rsbl.2010.0620 1744-957X), incorporated herein by reference in its entirety. Further, codon usage tables for strains of M. maripaludis are provided below:

Methanococcus maripaludis S2: 1722 CDS (492445 codons)
fields: [triplet] [frequency per thousand] ([number])

| | | | |
|---|---|---|---|
| UUU 32.7 (16089) | UCU 12.5 (6178) | UAU 20.2 (9944) | UGU 8.2 (4054) |
| UUC 9.2 (4527) | UCC 4.9 (2433) | UAC 17.9 (8800) | UGC 5.4 (2641) |
| UUA 38.3 (18884) | UCA 23.5 (11560) | UAA 3.0 (1466) | UGA 0.3 (135) |
| UUG 11.3 (5554) | UCG 4.3 (2102) | UAG 0.3 (130) | UGG 6.5 (3191) |
| CUU 25.6 (12589) | CCU 11.6 (5695) | CAU 6.3 (3105) | CGU 1.6 (800) |
| CUC 6.9 (3384) | CCC 3.0 (1465) | CAC 8.6 (4258) | CGC 0.5 (251) |
| CUA 4.9 (2427) | CCA 15.6 (7673) | CAA 9.4 (4633) | CGA 2.7 (1338) |
| CUG 3.9 (1940) | CCG 3.7 (1840) | CAG 9.2 (4549) | CGG 1.3 (641) |
| AUU 48.4 (23817) | ACU 16.9 (8313) | AAU 36.5 (17958) | AGU 10.3 (5049) |
| AUC 16.5 (8141) | ACC 7.3 (3585) | AAC 19.7 (9712) | AGC 6.9 (3384) |
| AUA 28.8 (14173) | ACA 19.4 (9535) | AAA 78.6 (38715) | AGA 17.8 (8788) |
| AUG 25.6 (12608) | ACG 6.0 (2934) | AAG 9.6 (4712) | AGG 6.9 (3378) |
| GUU 35.7 (17593) | GCU 14.1 (6949) | GAU 39.4 (19386) | GGU 16.5 (8140) |
| GUC 4.5 (2201) | GCC 3.5 (1714) | GAC 16.4 (8097) | GGC 8.3 (4103) |
| GUA 24.6 (12109) | GCA 35.8 (17620) | GAA 74.0 (36457) | GGA 35.2 (17322) |
| GUG 5.0 (2440) | GCG 4.5 (2222) | GAG 7.0 (3443) | GGG 7.3 (3571) |

Methanococcus maripaludis: 51 CDS (14512 codons)
fields: [triplet] [frequency per thousand] ([number])

| | | | |
|---|---|---|---|
| UUU 24.9 (362) | UCU 10.3 (150) | UAU 14.7 (213) | UGU |
| | | | 10.7 (156) |
| UUC 14.5 (210) | UCC 4.4 (64) | UAC 19.4 (282) | UGC 7.1 (103) |
| UUA 35.8 (519) | UCA 20.9 (304) | UAA 3.0 (43) | UGA 0.6 (9) |
| UUG 10.7 (156) | UCG 2.6 (38) | UAG 0.2 (3) | UGG 7.9 (115) |
| CUU 20.9 (304) | CCU 14.3 (208) | CAU 4.7 (68) | CGU 1.3 (19) |
| CUC 8.8 (128) | CCC 3.2 (47) | CAC 10.7 (156) | CGC 0.3 (4) |
| CUA 3.7 (53) | CCA 20.1 (291) | CAA 12.8 (186) | CGA 2.0 (29) |
| CUG 3.0 (43) | CCG 3.3 (48) | CAG 10.5 (153) | CGG 0.9 (13) |
| AUU 42.4 (615) | ACU 17.0 (247) | AAU 25.6 (371) | AGU |
| | | | 10.0 (145) |
| AUC 19.7 (286) | ACC 9.0 (130) | AAC 20.7 (300) | AGC 7.4 (107) |
| AUA 22.1 (321) | ACA 21.4 (310) | AAA 73.3 (1064) | AGA |
| | | | 21.6 (314) |
| AUG 29.4 (426) | ACG 5.3 (77) | AAG 9.5 (138) | AGG 7.2 (105) |
| GUU 36.9 (535) | GCU 18.9 (274) | GAU 35.3 (512) | GGU |
| | | | 22.5 (327) |
| GUC 4.3 (62) | GCC 3.2 (47) | GAC 20.1 (291) | GGC 8.2 (119) |
| GUA 27.6 (400) | GCA 43.8 (636) | GAA 73.1 (1061) | GGA |
| | | | 37.3 (542) |
| GUG 3.6 (52) | GCG 3.0 (44) | GAG 5.9 (85) | GGG 6.3 (92) |

Methanococcus maripaludis C5: 1822 CDS (515977 codons)
fields: [triplet] [frequency: per thousand] ([number])

| | | | |
|---|---|---|---|
| UUU 32.4 (16698) | UCU 12.2 (6314) | UAU 21.1 (10880) | UGU 8.2 (4218) |
| UUC 10.2 (5245) | UCC 5.2 (2667) | UAC 17.9 (9233) | UGC 4.9 (2530) |
| UUA 37.6 (19389) | UCA 23.1 (11932) | UAA 2.9 (1509) | UGA 0.3 (165) |
| UUG 13.0 (6717) | UCG 4.5 (2318) | UAG 0.3 (157) | UGG 6.5 (3339) |
| CUU 24.2 (12467) | CCU 10.8 (5562) | CAU 7.4 (3822) | CGU 1.7 (888) |
| CUC 7.7 (3980) | CCC 3.1 (1592) | CAC 7.8 (4042) | CGC 0.5 (250) |
| CUA 5.6 (2871) | CCA 15.4 (7942) | CAA 10.2 (5240) | CGA 3.0 (1544) |
| CUG 3.5 (1809) | CCG 3.8 (1948) | CAG 8.7 (4482) | CGG 1.3 (679) |
| AUU 47.6 (24550) | ACU 16.9 (8737) | AAU 36.6 (18869) | AGU 10.7 (5507) |
| AUC 17.6 (9099) | ACC 7.6 (3916) | AAC 19.5 (10068) | AGC 6.4 (3288) |
| AUA 28.8 (14858) | ACA 19.1 (9865) | AAA 78.1 (40308) | AGA 18.6 (9616) |
| AUG 25.6 (13218) | ACG 5.7 (2957) | AAG 11.0 (5688) | AGG 6.6 (3398) |
| GUU 34.5 (17783) | GCU 14.0 (7218) | GAU 40.1 (20716) | GGU 16.9 (8710) |
| GUC 4.7 (2441) | GCC 3.5 (1788) | GAC 16.0 (8251) | GGC 7.9 (4052) |
| GUA 23.9 (12331) | GCA 33.9 (17512) | GAA 74.7 (38536) | GGA 33.8 (17456) |
| GUG 5.2 (2658) | GCG 4.8 (2451) | GAG 7.6 (3903) | GGG 7.4 (3800) |

Methanococcus maripaludis C7: 1788 CDS (513919 codons)
fields: [triplet] [frequency: per thousand] ([number])

| | | | |
|---|---|---|---|
| UUU 32.1 (16506) | UCU 12.3 (6335) | UAU 20.7 (10621) | UGU 8.2 (4211) |
| UUC 10.2 (5230) | UCC 5.3 (2725) | UAC 18.0 (9247) | UGC 4.8 (2449) |
| UUA 36.2 (18601) | UCA 23.2 (11903) | UAA 2.9 (1494) | UGA 0.3 (161) |
| UUG 12.8 (6575) | UCG 4.6 (2369) | UAG 0.3 (142) | UGG 6.7 (3461) |
| CUU 24.6 (12633) | CCU 10.8 (5567) | CAU 6.9 (3538) | CGU 1.8 (900) |
| CUC 8.0 (4124) | CCC 3.0 (1545) | CAC 8.0 (4088) | CGC 0.5 (246) |
| CUA 5.6 (2890) | CCA 15.4 (7901) | CAA 9.7 (4980) | CGA 2.8 (1418) |
| CUG 3.8 (1946) | CCG 4.1 (2127) | CAG 9.1 (4677) | CGG 1.4 (712) |
| AUU 47.6 (24452) | ACU 17.2 (8815) | AAU 36.2 (18617) | AGU 10.8 (5558) |
| AUC 17.9 (9210) | ACC 7.6 (3902) | AAC 19.5 (10034) | AGC 6.5 (3365) |
| AUA 28.7 (14738) | ACA 19.4 (9981) | AAA 76.0 (39052) | AGA 17.9 (9224) |
| AUG 25.5 (13120) | ACG 6.0 (3101) | AAG 10.9 (5619) | AGG 6.9 (3528) |
| GUU 35.1 (18052) | GCU 14.3 (7357) | GAU 40.3 (20693) | GGU 17.1 (8767) |
| GUC 4.8 (2487) | GCC 3.6 (1852) | GAC 15.7 (8066) | GGC 7.7 (3949) |
| GUA 23.8 (12240) | GCA 34.5 (17723) | GAA 74.9 (38483) | GGA 34.6 (17792) |
| GUG 5.1 (2607) | GCG 4.8 (2491) | GAG 7.5 (3875) | GGG 7.5 (3847) |

### Source Organisms

In some embodiments the source organism is an anaerobic methanotroph. In some embodiments, the source organism is a member of the domain Archaea that is an anaerobic methanotroph (“ANME”). ANME Group 1, ANME Group 2, and ANME Group 3 (ANME-1, ANME-2, and ANME-3, respectively) are three currently recognized groups of anaerobic methanotrophic archaea. The ANME organisms currently have no technical classification, though they are members of the Euryarchaeota, e.g., in the class Methanomicrobia. ANME-1 is distantly related to the Methanosarcinales and Methanomicrobiales; ANME-2 are organisms related to organisms belonging to the order Methanosarcinales.

In some embodiments, the source organism is a member of the taxon Euryarchaeota. In some embodiments, the source organism is a member of the taxon Methanobacteria. In some embodiments, the source organism is a member of the taxon Methanobacteriales. In some embodiments, the source organism is a member of the taxon Methanobacteriaceae. In some embodiments, the source organism is a member of the genus Methanothermobacter. For example, in some embodiments the source organism is the species Methanothermobacter marburgensis. In some embodiments, the source organism is a strain of the species M. marburgensis, such as Methanothermobacter marburgensis str. Marburg.

In some embodiments, the source organism is a member of the taxon Methanomicrobia. In some embodiments, the source organism is a member of the taxon Methanosarcinales. In some embodiments, the source organism is a member of the taxon Methanosarcinaceae. In some embodiments, the source organism is a member of the genus Methanosarcina. In some embodiments, the source organism is a species Methanosarcina barkeri. In some embodiments, the source organism is a strain of Methanosarcina barkeri that is, e.g., Methanosarcina barkeri 227, Methanosarcina barkeri 3, Methanosarcina barkeri CM1, Methanosarcina barkeri JCM 10043, Methanosarcina barkeri MS, Methanosarcina barkeri str. Fusaro, or Methanosarcina barkeri str. Wiesmoor. In some embodiments, the source organism is Methanosarcina acetivorans.

In some embodiments, the source organism is a member of the taxon Methanococci. In some embodiments, the source organism is a member of the taxon Methanococcales. In some embodiments, the source organism is a member of the taxon Methanococcaceae. In some embodiments, the source organism is a member of the genus Methanococcus. In some embodiments, the source organism is a species Methanococcus voltae. In some embodiments, the source organism is a strain of Methanococcus voltae, e.g., Methanococcus voltae A3 or Methanococcus voltae PS. In some embodiments, the source organism is a member of the genus Methanocaldococcus. In some embodiments, the source organism is a species Methanocaldococcus jannaschii. In some embodiments, the source organism is a strain of Methanocaldococcus jannaschii, e.g., Methanocaldococcus jannaschii DSM 2661.

In some embodiments, the source organism is related to M. maripaludis, e.g., Methanothermococcus thermolithotrophicus or Methanococcus aeolicus, which is a mesophilic relative of M. maripaludis. Accordingly, in some embodiments the source organism is Methanothermococcus thermolithotrophicus (e.g., a strain of Methanothermococcus thermolithotrophicus that is Methanothermococcus thermolithotrophicus DSM 2095) or Methanococcus aeolicus (e.g., a strain of Methanococcus aeolicus that is Methanococcus aeolicus Nankai-3).

In some embodiments, the source organism comprises one or more enzymes, genes, or pathways for producing solvent. For example, in some embodiments the source organism is a solvent-producing bacterium such as a member of the clostridia, e.g., that possesses a Wood-Ljungdahl pathway and/or a metabolic pathway for butanol production. Accordingly, in some embodiments, the source organism is a member of the domain Bacteria. In some embodiments, the source organism is a member of the taxon Firmicutes. In some embodiments, the source organism is a member of the taxon Clostridia. In some embodiments, the source organism is a member of the taxon Clostridiales. In some embodiments, the source organism is a member of the taxon Clostridiaceae. In some embodiments, the source organism is a member of the genus Clostridium. In some embodiments, the source organism is a species Clostridium carboxidivorans (e.g., a Clostridium carboxidivorans strain that is Clostridium carboxidivorans P7) or a species Clostridium acetobutylicum (e.g., a. Clostridium acetobutylicum strain that is Clostridium acetobutylicum ATCC 824, Clostridium acetobutylicum DSM 1731, or Clostridium acetobutylicum EA 2018).

### Host Microorganisms

Embodiments provide for the expression of metabolic pathways, genes, and gene modules from one or more source organism(s) in a heterologous host microorganism. Accordingly, in some preferred embodiments, the technology relates to use of a genetically tractable host microorganism, e.g., a host microorganism that is culturable and amenable to genetic manipulation. One of skill in the art can select suitable culture media and growth conditions for the host microorganism. See, e.g., Farkas et al. (2013) “Genetic Techniques for the Archaea”, Annual Review of Genetics 47: 539-561, incorporated herein by reference in its entirety.

An exemplary host microorganism is Methanococcus maripaludis, which is a well-developed hydrogenotrophic methanogen model organism (see, e.g., Leigh et al (2011) FEMS Microbiol Rev 35: 577-608). Further, the complete genome sequence of M. maripaludis is known (see, e.g., Hendrickson et al (2004) “Complete genome sequence of the genetically tractable hydrogenotrophic methanogen Methanococcus maripaludis”, J Bacteriol. 186(20): 6956-69). The wild type strain of M. maripaludis is known as M maripaludis S2 (which is also referred to as LL) is available at the Deutsche Sammlung von Mikroorganismen and Zellkulturen as DSM 14266. In some embodiments, M. maripaludis cultures are grown with under hydrogen and carbon dioxide gases (e.g., at 200 to 400 kPa, e.g., at approximately 276 kPa of H₂/CO₂ gas at a 80:20 ratio vol/vol) at 37° C. in McN (mineral medium), in McNA (McN plus 10 mM sodium acetate), or in McC (complex medium minus vitamin solution) as described previously in Whitman et al. (1986), “Isolation and characterization of 22 mesophilic methanococci”, System. Appl. Microbiol. 7: 235-40, incorporated herein by reference in its entirety. In some embodiments, for preparation of cell free extracts, M. maripaludis strains are grown in bottles with approximately 50 to 500 ml (e.g., 100 ml) McNA medium under approximately 100 to 200 kPa hydrogen and carbon dioxide gases (e.g., 138 kPa H₂/CO₂ gas at a ratio of approximately 80:20 vol/vol) at 37° C. In some embodiments, M. maripaludis is grown in a defined complex medium (McA) as described in Haydock et al (2004) “Continuous culture of Methanococcus maripaludis under defined nutrient conditions” FEMS Microbiol. Lett. 238:85-91. In some embodiments, M. maripaludis is grown in a system for steady-state growth in chemostats as described in Haydock et al (2004) FEMS Microbiol Lett 238: 85-91, incorporated herein by reference.

Like most methanogens, M. maripaludis grows under strictly anaerobic, reduced conditions. For example, most methanogens are grown on hydrogen and carbon dioxide as their primary substrates for energy-generating metabolism. M. maripaludis provides several advantages for the present technology, including rapid and facile growth under laboratory conditions and robust techniques for genetic manipulation. In particular, M. maripaludis has a doubling time of approximately two hours at a growth temperature of 37° C. Accordingly, M. maripaludis produces stationary phase liquid cultures overnight and colonies are produced on agar medium in two or three days. As a result, experiments progress rapidly. Further, metabolic flux is rapid. M. maripaludis can be grown in a fermenter for large-scale cultivation and chemostats can be used for continuous culture (see, e.g., Haydock et al (2004) FEMS Microbiol Lett 238: 85-91, incorporated herein by reference). Continuous culture techniques provide for both the rigorous and reproducible control of nutritional conditions and for eliminating or minimizing variations in growth rate and cell density.

Genetic manipulations in some Archaea are traditionally difficult due to their non-susceptibility to many antibiotics used for manipulation of Bacteria and their specialized growth conditions. However, genetic manipulations in M. maripaludis are facilitated by a high-efficiency transformation procedure, the use of two effective antibiotic selections (e.g., using puromycin and neomycin), and the use of 6-azauracil for counterselection. These tools provide the ability to use a “pop-in pop-out” approach to make markerless genetic mutations consisting of in-frame deletions, insertions, or other kinds of mutations that are not encumbered by residuals such as antibiotic resistance markers. For example, M. maripaludis has been used to produce an organism containing seven different in-frame deletion mutations and an additional mutation that induced the expression of an otherwise cryptic pathway (see, e.g., Costa et al (2013) MBio 4).

In additional embodiments, the host organism is a species of Methanosarcina, which are amenable to genetic manipulation but which also grow much more slowly than M. maripaludis.

Accordingly, in some embodiments, the host microorganism is a methanogen. In some embodiments, the host microorganism is an anaerobic methane oxidizer. In some embodiments, the host microorganism is a member of the Bacteria, Archaea, or Eukarya. In some embodiments, the host microorganism is a member of the Euryarchaeota. In some embodiments, the host microorganism is a member of the Methanococci. In some embodiments, the host microorganism is a member of the Methanococcales. In some embodiments, the host microorganism is a member of the Methanococcaceae. In some embodiments, the host microorganism is a member of the genus Methanococcus. In some embodiments, the host microorganism is the species Methanococcus maripaludis. In some specific embodiments, the host microorganism is a strain of the species Methanococcus maripaludis, e.g., M. maripaludis C5, M. maripaludis C6, M. maripaludis C7, M. maripaludis S2, or M. maripaludis X1.

In some embodiments, the host microorganism is a member of the Methanomicrobia; in some embodiments, the host microorganism is a member of the Methanosarcinales; in some embodiments, the host microorganism is a member of the Methanosarcinaceae. In some embodiments, the host microorganism is a member of the genus Methanosarcina. In some embodiments, the host microorganism is the species Methanosarcina acetivorans, Methanosarcina baltica, Methanosarcina barkeri, Methanosarcina calensis, Methanosarcina horonobensis, Methanosarcina lacustris, Methanosarcina mazei, Methanosarcina semesiae, Methanosarcina sicibae, Methanosarcina soligelidi, Methanosarcina spelaei, Methanosarcina thermophila, or Methanosarcina vacuolata.

Some embodiments provide a host microorganism that has undergone strain adaptation under selective conditions to identify variants with improved properties for production. Improved properties may include increased growth rate, improved yield of desired products, and increased tolerance of process contaminants.

### Vectors

Further, several recombinant vectors are available that provide for the heterologous expression of exogenous nucleic acids in M. maripaludis, e.g., for the heterologous expression of anaerobic proteins as described herein. Further, the recombinant vectors provide for the generation of site specific variants. The vectors can integrate into the genome or can remain as separate epigenomic replicons. Some vectors comprise antibiotic selection markers, promoters for the expression of cloned genes, and oligo-his tag moieties for the tagging of proteins. These vectors include multicopy replicative vectors and integrative vectors that insert into the genome in single copy. Strong promoters are present to drive expression of recombinant genes. Some promoters are constitutive (e.g., histone and S-layer promoters) and some promoters are inducible by starvation for nitrogen (e.g., nif promoter). Some vectors comprise an antibiotic resistance markers such genes providing resistance to puromycin (pur) and/or genes providing resistance to neomycin (neo). Some vectors comprise an oligo-his moiety for tagging a protein on its N-terminus or C-terminus.

TABLE 1
vectors

| | | antibiotic | |
|---|---|---|---|
| | promoter | marker | features |
| Replicative | | | |
| pWLG series | histone promoter | pur or neo | N- or C-terminal his tag |
| pMEV1.1.2 | S-layer promoter | pur | |
| pHW40 | nif promoter | pur | |
| Integrative | | | |
| pJK3 | Methanosarcina | pur | site for insertion of |
| | mcr promoter | | homologous sequence |
| pIJA03NH | various | neo | integrates into argH |
| pBLprt | various | neo or | integrates into upt |
| | | markerless | |

These vectors (e.g., as shown in Table 1) have been used for expressing genes in M. maripaludis, e.g., for genetic manipulation, genetic complementation, and/or for production of active proteins for biochemical or structural studies. During experiments conducted during the development of technologies described herein, M. maripaludis gene expression strains were grown in fermenters and oligo-his tagged proteins were expressed and purified (e.g., using a purification pipeline featuring anaerobic Ni-affinity chromatography and an FPLC housed inside an anaerobic chamber). As an example of the genetic manipulability and tractability of M. maripaludis, experiments were conducted to perform biochemical studies of M. maripaludis nitrogenase and nitrogenase inhibitor NifI using protein expressed in M. maripaludis (see, e.g., Dodsworth & Leigh (2006) Proc Natl Acad Sci USA 103: 9779-84); and experiments were conducted to express, purify, and determine the crystal structure of a Methanocaldococcus sp. NifI heterologously expressed in M. maripaludis. During the development of embodiments of the technology described herein, a yield of approximately 1 to 2 mg of heterologously expressed protein was obtained per one gram of cell dry weight. Further, experiments conducted during the development of the technology provided herein used genetic manipulation of M. maripaludis to express in M. maripaludis, and subsequently to purify, heterodisulfide reductases (HDR) from both M. maripaludis and the methanogen Methanothermobacter marburgensis. These experimental results demonstrate the robust tractability of M. maripaludis as a host microorganism and the potential for genetic manipulation of M. maripaludis to express a variety of products from heterologous source organisms.

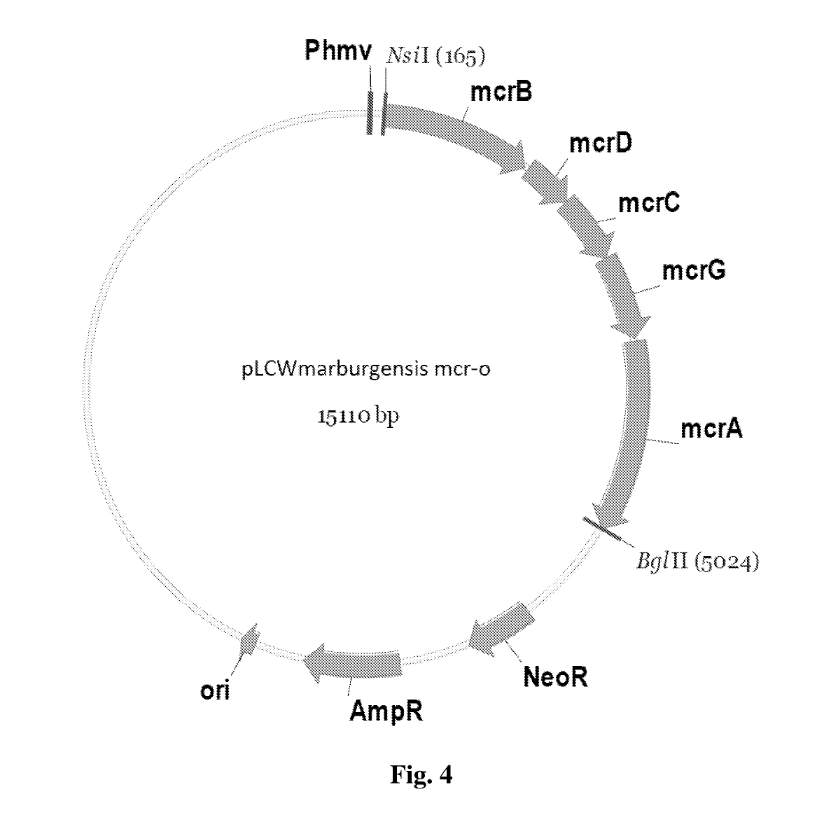

In some embodiments of the technology, a vector (e.g., a plasmid) comprises the genes for an MCR. For instance, some embodiments provide a plasmid expressing the mcrB, mcrD, mcrC, mcrG, and/or mcrA genes (see, e.g., FIG. 4). In some embodiments, the mcrB, mcrD, mcrC, mcrG, and/or mcrA genes are from Methanothermobacter marburgensis (see, e.g., FIG. 4). In some embodiments, the mcrB, mcrD, mcrC, mcrG, and/or mcrA genes are from an ANME organism such as ANME-2 (e.g., ANME-2c). In some embodiments, the plasmid vector is based on the pWLG vector (see, e.g., Table 1). Embodiments related to vectors comprising genes encoding MCR provide vectors comprising appropriate promoters and other associated regulatory elements, enhancers, repressors, operators, etc. for heterologous expression in a host organism. Some exemplary regulatory elements, enhancers, repressors, operators, etc. for heterologous expression in a host organism include, e.g., a Methanococcus voltae histone (e.g., hmv) promoter, a Methanococcus voltae mcr, promoter, a Methanococcus voltae S-layer promoter, a Methanococcus maripaludis (nitrogen fixation) nif promoter, a Methanococcus maripaludis (nitrogen fixation) nif operator, a lac operator, a lac repressor, a tet operator, and a tet repressor.

### Non-Naturally Occurring Microorganisms

In certain embodiments, the technology provides a non-naturally occurring microorganism that is genetically engineered to comprise an exogenous nucleic acid encoding an enzyme with coenzyme M reductase activity. In some embodiments, the non-naturally occurring microorganism is produced by introducing an exogenous nucleic acid from a non-culturable source organism into a culturable host strain, e.g., to provide a culturable microorganism that comprises an exogenous nucleic acid encoding an enzyme with coenzyme M reductase activity. Such a culturable non-naturally occurring microorganism finds use, e.g., in producing an alcohol from an alkane (e.g., methanol from methane). In some embodiments, the non-naturally occurring microorganism is produced by “transformation”, which refers to the transfer of a nucleic acid (e.g., an exogenous nucleic acid) into the genome of a host microorganism, resulting in genetically stable inheritance. Host microorganisms containing the transformed nucleic acid are referred to as “non-naturally occurring”, “recombinant”, “transformed”, “genetically engineered”, or “transgenic” microorganisms. Host microorganisms may be selected from, and the non-naturally occurring microorganisms generated in, any prokaryotic or eukaryotic microbial species from the domains of Archaea, Bacteria, or Eukarya.

Accordingly, in some embodiments, the microorganism is capable of being cultured. In particular embodiments, the microorganism can be grown under controlled environmental conditions, e.g., in a defined synthetic medium. In some embodiments, the microorganism is grown at a controlled temperature, pressure, and volume. In some embodiments, the microorganism can be grown in a semi-defined medium. In embodiments, the microorganism is grown in co-culture with one or more naturally occurring or non-naturally occurring microorganisms.

Due to the broad specificity of some enzymes with coenzyme M reductase activity, non-naturally occurring microorganisms according to any of the embodiments disclosed herein may also be capable of converting ethane, propane, and butane into their corresponding alcohols, ethanol, propanol, and butanol, respectively.

In some embodiments, the non-naturally occurring organism comprise one or more genetic alterations, e.g., to optimize the heterologous pathways and enzymes in their new host. In some embodiments, the genetic alteration is a knock-out.

Non-naturally occurring microorganisms as described herein may be transformed to comprise at least one exogenous nucleic acid to provide the host organism with a new or enhanced activity (e.g., a methyl coenzyme M reductase activity) or may be genetically modified to remove or substantially reduce an endogenous gene function (e.g., methylene-H₄MPT reductase gene (mer)) using a variety of methods known in the art. Recombinant methods for exogenous expression of nucleic acids in microbial organisms are well known in the art. Such methods can be found described in, for example, Sambrook et al, Molecular Cloning: A Laboratory Manual, Third Ed., Cold Spring Harbor Laboratory, New York (2001); and Ausubel et al, Current Protocols in Molecular Biology, John Wiley and Sons, Baltimore, Md. (1999).

Non-naturally occurring microorganisms according to any of the embodiments disclosed herein may be provided as living whole cells (e.g., in culture, on a biofilm, or as a moist cell paste), non-living whole cells (e.g., lyophilized whole cell preparations, reconstituted whole cell preparations of lyophilized cells), or cell-free fractions derived thereof (e.g., lysates, membrane fractions). Enzyme activity may be separated from whole cells by association with a particular organelle, allowing use of cell-free fractions resulting from differential centrifugation (see, e.g., Nguyen et al, 1994, J. Biol. Chem. 269: 14995-15005; Scott and Higgins, 1981, Microbiol. 125:63-72). Cell-free fractions may include cell lysates and soluble or membrane fractions of centrifuged cell-free extracts. Cell-free fractions may also include protein extracts containing an enzyme, such as detergent-solubilized fractions (see, e.g., Smith and Dalton, 1989, Eur. J. Biochem. 182:667-671) or purified enzyme (see, e.g., Lieberman et al, 2003, Proc. Natl. Acad. Sci. USA 100:3820-3825). Whole cells and cell-free fractions may be provided in a substantially non-aqueous state (e.g., lyophilized). Lyophilized preparations of enzymes have been shown to retain catalytic activity (see, e.g., U.S. Patent Publication 2002/0168733; Nguyen et al, 1994, J. Biol. Chem. 269: 14995-15005).

In some embodiments, non-naturally occurring microorganisms according to any of the embodiments disclosed herein or cell-free fractions derived thereof are exposed to a gas substrate comprising methane, which provides a methane substrate for conversion into methanol. A gas substrate comprising methane may be a substantially purified form, such as pipeline quality natural gas. In certain embodiments, a gas substrate comprising methane is a mixture of gases, such as wet natural gas. A gas mixture may comprise light alkanes (e.g., any combination of two or more alkanes selected from the group consisting of methane, ethane, propane, and butane).

In certain embodiments, methods described herein use non-naturally occurring microorganisms according to any of the embodiments disclosed herein or cell-free fractions derived thereof immobilized on, within, or behind a solid matrix. In further embodiments, the non-naturally occurring microorganisms or cell-free extracts derived thereof are in a substantially non-aqueous state (e.g., lyophilized). In some embodiments, non-naturally occurring microorganisms or cell-free fractions derived thereof are temporarily or permanently attached on, within, or behind a solid matrix within a bioreactor. Nutrients, substrates, and other required factors are supplied to the solid matrices so that the cells catalyze desired reactions. Non-naturally occurring microorganisms may grow on the surface of a solid matrix (e.g., as a biofilm). Non-naturally occurring microorganisms or cell-free fractions derived thereof may be attached on the surface or within a solid matrix without cellular growth or in a nonliving state. Exemplary solid matrix supports for microorganisms include polypropylene rings, ceramic bio-rings, ceramic saddles, fibrous supports (e.g., membrane), porous glass beads, polymer beads, charcoal, activated carbon, dried silica gel, particulate alumina, Ottawa sand, clay, polyurethane cell support sheets, and fluidized bed particle carrier (e.g., sand, granular-activated carbon, diatomaceous earth, calcium alginate gel beads).

### Research Uses

In some embodiments, the technology finds use in research. For example, in some embodiments the technology provides a culturable microorganism comprising an exogenous nucleic acid encoding an enzyme with coenzyme M reductase activity for study and analysis in a laboratory environment, e.g., to provide a system to analyze the anaerobic oxidation of methane. In some embodiments, the technology provides a culturable microorganism comprising an exogenous nucleic acid encoding an enzyme with coenzyme M reductase activity for computational modeling of metabolic flux in an organism. For example, in some embodiments the genetically engineered microorganisms as described herein are studied using a genome-scale flux balance model to assess methane oxidation in an anaerobic methanogen and describe the mass and energy balances for these systems. In some embodiments, this model finds use in modulating the metabolic network of the genetically engineered microorganisms so that the genetically engineered pathways are utilized efficiently within the constraints of the organism's metabolic needs. Such an approach is valuable in utilizing the genetically engineered organisms for the transformation of methane, as these organisms provide one-carbon metabolism for both fuel production and fuel utilization. In some embodiments, the genetically engineered microorganism and genome-scale metabolic modeling of the genetically engineered organism (e.g., a M. maripaludis, e.g., a M. maripaludis strain) comprises the ANME genes for anaerobic methane oxidation and the other genes needed to convert methane to methanol and to butanol.

In some embodiments, a genetically engineered microorganism comprising a host that is Methanococcus maripaludis and a heterologous methyl coenzyme M reductase from a source microorganism that is a group 1 anaerobic methanotroph (ANME-1), a group 2 anaerobic methanotroph (ANME-2), or a group 3 anaerobic methanotroph (ANME-3) finds use in research as a platform into which to introduce and study additional metabolic capabilities such as a methanol:coenzyme M methyltransferase (e.g., from a source organism from the genus Methanosarcina, e.g., a source organism that is Methanosarcina barkeri), a dissimilatory sulfate reduction pathway (e.g., comprising one or more of a heterodisulfide reductase, an ATP sulfurylase, an APS reductase, a sulfite reductase, and/or a hydrogenase), electron transport proteins and/or cofactors, a pathway for sulfite reduction comprising an assimilatory sulfite reductase, a pathway for converting methanol into butanol, a Wood-Ljungdahl pathway, a pathway for production of butanol from acetyl-CoA. In some embodiments, the genetically engineered microorganism provides a model to research the metabolic pathways of an organism further comprising an inactivated mer gene.

### EXAMPLES

### Example 1

### Expression of Methanothermobacter marburgensis and ANME-2c Mcr Proteins in Methanococcus maripaludis

During the development of embodiments of the technology provided herein, experiments were conducted to express Methanothermobacter marburgensis and ANME-2c Mcr proteins in Methanococcus maripaludis.

### Materials and Methods

The pLCW40 and pHW40 plasmids were used to clone and express mcr genes. Plasmid pLCW40 contains the Methanococcus voltae hmv promoter and a hexahistidine sequence (see e.g., Dodsworth & Leigh (2006) “Regulation of nitrogenase by 2-oxoglutarate-reversible, direct binding of a PII-like nitrogen sensor protein to dinitrogenase” Proc. Natl. Acad. Sci USA 103: 9779-84). pHW40 contains the Methanococcus maripaludis nif promoter (see, e.g., VanDyke et al. (2008) “Identification of a putative acetyltransferase gene, MMP0350, which affects proper assembly of both flagella and pili in the archaeon Methanococcus maripaludis” J Bacteriol. 190: 5300-07).

Plasmid pIJA03LCW was constructed as follows. The hmv promoter (Pₕₘᵥ) and the hexahistine (H₆) portions of pLCW40 were amplified separately using pLCW40 as template and the following primers:

Pₕₘᵥ primers:
forward primer

SEQ ID NO: 1

GGGAGCTCGAGATAAGAATTACTAGTTAATTC
reverse primer

SEQ ID NO: 2

AAGCGGCCGCCCATGCATTTCACCTATTAGTTATC
H₆ primers:
forward primer

SEQ ID NO: 3

AAGCGGCCGCATCGAAGGTCGTCATCACCATCAC
reverse primer

SEQ ID NO: 4

GTTTATATATAAGATGTTAATAACACAG

Underlined sequences are sites recognized by XhoI and NotI restriction enzymes in the Pₕₘᵥ forward primer (XhoI), the Pₕₘᵥ reverse primer (NotI), and H₆ forward primer (Nod).

The products were mixed, digested with NotI, and ligated together. The product was then amplified and digested with XhoI and XbaI. The digested fragment was cloned into pIJA03 (see, e.g., Stathopoulos et al (2001) “Cysteinyl-tRNA synthetase is not essential for viability of the archaeon Methanococcus maripaludis” Proc Natl Acad Sci USA 98: 14292-97) that was also digested with XhoI and XbaI to produce pIJA03LCW.

The complete mcrBDCGA operons from Methanothermobacter marburgensis and the ANME-2C strain were synthesized de novo and codon optimized for high expression in the host strain Methanococcus maripaludis. Gene synthesis and codon optimization were aided by software tools (e.g., Genewiz, 115 Corporate Boulevard, South Plainfield, N.J. 07080). Additionally, a hexahistidine tag was added to the C-terminal coding sequence of the last gene of each operon. The codon-optimized synthetic operons were cloned in E. coli plasmid vector pUC57-Kan.

Then, the hexahistidine-tagged M. marburgensis operon was amplified from the pUC57-Kan template using the following primers:

forward primer

SEQ ID NO: 5

AAATGCATGGCAAAATTTGAGGACAAAGTTGACC
reverse primer

SEQ ID NO: 6

AAAGATCTTTAGTGATGGTGATGGTGATGACGACCTTCG

Underlined sequences are sites recognized by NsiI and BglII restriction enzymes in the forward primer (NsiI) and reverse primer (BglII).
The ANME-2C operon was first cloned into pIJA03LCW digested with NsiI and NotI. This construct was used as the template for amplification of the hexahistidine-tagged operon using the following primers:

forward primer

SEQ ID NO: 7

AAATGCATGGCTGACAAGATTGATTTATATGATG
reverse primer

SEQ ID NO: 8

AAAGATCTTAGTGATGGTGATGGTGATGACGACCTTCG

Underlined sequences are sites recognized by NsiI and BglII restriction enzymes in the forward primer (NsiI) and reverse primer (BglII).

The M. marburgensis Mcr amplification product was then digested with NsiI and BglII and cloned into pLCW40 digested with NsiI and BglII (see FIG. 4). In this vector construct, the Methanococcus voltae hmv promoter drives the constitutive expression of the inserted M. marburgensis mcr genes (FIG. 4, Pₕₘᵥ).

The ANME-2C Mcr amplification product was then digested with NsiI and BglII and cloned into pHW40 digested with NsiI and BglII. In this similar construct in which the mcr genes from ANME-2c were cloned into the pLCW vector, the nif promoter drives expression of the ANME-2c mcr genes.

The vectors comprise an ampicillin resistance gene (FIG. 4, ampR) and a neomycin resistance gene (FIG. 4, neoR), e.g., for selection and/or screening of transformed microorganisms.

The plasmid constructs were transformed into M. maripaludis by PEG-mediated transformation (see, e.g., Tumbula et al (1994) “Transformation of Methanococcus maripaludis and identification of a PstI-like restriction system” FEMS Microbial. Lett. 121: 309-14) and transformants were grown under selection using puromycin. Cultures were grown in fermenters in defined medium as described in Haydock et al (2004) “Continuous culture of Methanococcus maripaludis under defined nutrient conditions” FEMS Microbiol. Lett. 238: 85-91, except that amino acids, purines, pyrimidines, and organic acids except acetate were omitted. The nitrogen source was ammonium for growing transformants expressing the M. marburgensis Mcr. The nitrogen source was N₂ for growing transformants expressing the ANME-2C Mcr.

Mcr proteins were purified from cell extracts anaerobically by Ni-affinity chromatography (see, e.g., Dodsworth & Leigh (2006) “Regulation of nitrogenase by 2-oxoglutarate-reversible, direct binding of a PII-like nitrogen sensor protein to dinitrogenase” Proc. Natl. Acad. Sci. USA 103: 9779-84) and protein concentrations were measured by the Bradford assay. Mcr from M. marburgensis was expressed at 0.50 mg protein/g cell dry weight and Mcr from ANME-2c was expressed at 0.20 mg protein/g cell dry weight.

### Example 2

### Heterologous Expression of Methanosarcina acetivorans Methanol Methyltransferase

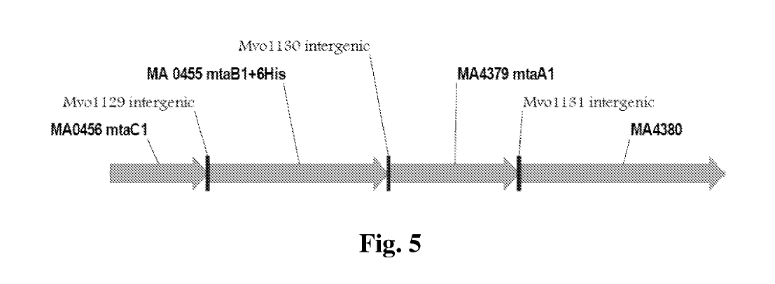

During the development of embodiments of the technology described herein, experiments were conducted to express a methanol methyltransferase (e.g., a multi-subunit methanol methyltransferase) in a heterologous host organism. In particular, the methanol methyltransferase mtaC, mtaB, and mtaA genes from Methanosarcina acetivorans were cloned and used to construct a synthetic operon in a replicative plasmid vector (see, e.g., FIG. 5) for expression in a heterologous host, e.g., Methanococcus maripaludis. In some embodiments, a nucleotide sequence encoding a histidine tag (e.g., a 6-histidine tag) is added to the 3′ end of the mtaB gene, e.g., to encode an MtaB protein comprising a 6-histidine tag at its C-terminus. Furthermore, in some embodiments, the synthetic operon further comprises a gene encoding a putative activator protein (MA4380) that is involved in reducing the cobalt cofactor in the corrinoid center of the multisubunit methanol methyltransferase enzyme (see, e.g., FIG. 5). Accordingly, the synthetic operon comprises nucleotide sequences encoding MtaC, MtaB (e.g., with a C-terminal histidine tag), MtaA, and the activator MA4380.

In addition, in some embodiments the synthetic operon comprises intergenic sequences from Methanococcus voltae (see, e.g., FIG. 5, “Mvo1129 intergenic”, “Mvo1130 intergenic”, “Mvo1131 intergenic”) and, in some embodiments, the genes in the operon are codon optimized for expression in the heterologous host (e.g., Methanococcus maripaludis).

Furthermore, in some embodiments, the construct comprises a promoter to control expression of the genes in the synthetic operon; for example, in some embodiments, a Methanococcus voltae constitutively expressed histone promoter (e.g., the hmv promoter) is used to modulate (e.g., drive) expression of the genes in the synthetic operon (see, e.g., Gardner and Whitman (1999), Genetics 152(4): 1439-47, incorporated herein by reference). And, in some embodiments, the construct comprises a transcriptional terminator; for example, in some embodiments, the construct comprises a transcriptional terminator from the Methanococcus maripaludis S-layer gene. Without being bound by theory, it is contemplated that including intergenic sequences from a related organism and a transcriptional terminator improves mRNA levels.

FIG. 5 shows an exemplary embodiment of the synthetic operon construct comprising four Mta genes (mtaC1, mtaB1+histidine tag), mtaA1, MA4380) with intergenic sequences from Methanococcus voltae. The genes are codon-optimized and the sequence encoding the MtaB subunit comprises a nucleotide sequence encoding a histidine tag on the C-terminal end. The synthetic operon was assembled into a replicative plasmid vector following the Methanococcus voltae hmv promoter and a transcriptional terminator from the Methanococcus maripaludis S-layer gene follows the synthetic operon.

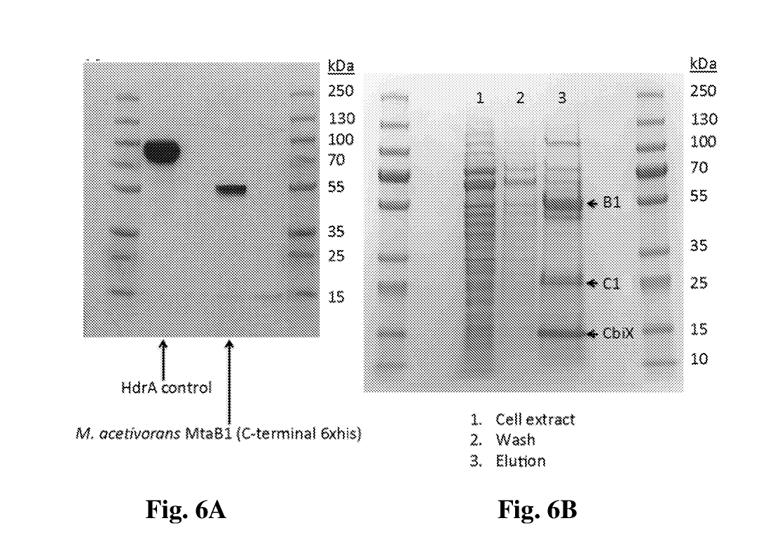

Data were collected from experiments testing the expression of the Methanosarcina acetivorans methanol methyltransferase in Methanococcus maripaludis (see, e.g., FIG. 6A and FIG. 6B). The data indicated significant expression of the methanol methyltransferase proteins (see, e.g., FIG. 6A). In particular, expression of the MtaB1 subunit was similar to the expression of HdrA control, which is known to be expressed at a high level. The data further indicated that the MtaB and MtaC subunits were purified in a single histidine tag pull-down step, e.g., using a nickel affinity column and elution by imidazole. The data indicated that the MtaC subunit co-purifies with the histidine-tagged MtaB subunit (see, e.g., FIG. 6B).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in the art are intended to be within the scope of the following claims.

We claim:

1. A genetically engineered microorganism comprising a host microorganism and a heterologous methyl coenzyme M reductase and/or a heterologous methanol methyl transferase.

2. The genetically engineered microorganism of claim 1 wherein the host microorganism is from the domain Archaea and/or is a methanogen.

3. The genetically engineered microorganism of claim 1 wherein the host microorganism is from the genus Methanococcus.

4. The genetically engineered microorganism of claim 1 wherein the host microorganism is Methanococcus maripaludis.

5. The genetically engineered microorganism of claim 1 wherein the heterologous methyl coenzyme M reductase is from a source microorganism that is from the domain Archaea and/or is an anaerobic methanotroph.

6. The genetically engineered microorganism of claim 1 wherein the heterologous methyl coenzyme M reductase is from a source microorganism that is a group 2 anaerobic methanotroph (ANME-2) or that is from the genus Methanothermobacter.

7. The genetically engineered microorganism of claim 1 wherein the heterologous methyl coenzyme M reductase is from a source microorganism that is Methanothermobacter marburgensis.

8. The genetically engineered microorganism of claim 1 wherein the heterologous methyl coenzyme M reductase is encoded by mcrB, mcrD, mcrC, mcrG, and mcrA genes.

9. The genetically engineered microorganism of claim 1 wherein the heterologous methanol methyl transferase is from a source microorganism that is from the domain Archaea and/or is a methanogen.

10. The genetically engineered microorganism of claim 1 wherein the heterologous methanol methyl transferase is from a source microorganism that is from the genus Methanosarcina.

11. The genetically engineered microorganism of claim 1 wherein the heterologous methanol methyl transferase is from a source microorganism that is Methanosarcina acetovorans.

12. The genetically engineered microorganism of claim 1 comprising an integrated and/or episomal vector comprising an mcr operon and/or an mta operon.

13. The genetically engineered microorganism of claim 1 comprising:
a) an integrated and/or episomal vector comprising mcrB, mcrD, mcrC, mcrG, and/or mcrA; and/or
b) an integrated and/or episomal vector comprising mtaC, mtaB, and/or mtaA.

14. A method for producing a fuel or a fuel mixture, the method comprising:
exposing a composition comprising an alkane to a genetically engineered microorganism comprising a host microorganism and a heterologous methyl coenzyme M reductase and/or a heterologous methanol methyl transferase to produce a fuel; and/or
exposing a composition comprising an alkane to a cell-free fraction derived from a genetically engineered microorganism comprising a host microorganism and a heterologous methyl coenzyme M reductase and/or a heterologous methanol methyl transferase to produce a fuel.

15. The method of claim 14 wherein the fuel or fuel mixture comprises methanol, ethanol, propanol, and/or butanol.

16. The method of claim 14 further comprising mixing the fuel with a petroleum-based fuel.

17. The method of claim 14 further comprising mixing the fuel with gasoline.

18. The method of claim 14 wherein the alkane is methane.

19. A fuel produced by a method comprising:
exposing a composition comprising an alkane to a genetically engineered microorganism comprising a host microorganism and a heterologous methyl coenzyme M reductase and/or a heterologous methanol methyl transferase to produce a fuel; and/or
exposing a composition comprising an alkane to a cell-free fraction derived from a genetically engineered microorganism comprising a host microorganism and a heterologous methyl coenzyme M reductase and/or a heterologous methanol methyl transferase to produce a fuel.

20. The fuel of claim 19 wherein the method further comprises mixing the fuel with a petroleum-based fuel.
